(19) United States
(12) Patent Application Publication
(10) Pub. No.: US 2013/0237689 A1
(43) Pub. Date: Sep. 12, 2013

(54) SOLUBLE HLA CLASS II COMPLEXES AND METHODS OF PRODUCTION AND USE THEREOF

(71) Applicants: William H. Hildebrand, Edmond, OK (US); Steven Cate, Moore, OK (US)

(72) Inventors: William H. Hildebrand, Edmond, OK (US); Steven Cate, Moore, OK (US)

(73) Assignee: THE BOARD OF REGENTS OF THE UNIVERSITY OF OKLAHOMA, Norman, OK (US)

(21) Appl. No.: 13/859,811

(22) Filed: Apr. 10, 2013

Related U.S. Application Data
(63) Continuation of application No. 12/859,002, filed on Aug. 18, 2010.
(60) Provisional application No. 61/234,937, filed on Aug. 18, 2009.
(60) Provisional application No. 61/333,827, filed on May 12, 2010.

(51) Int. Cl. C07K 14/74; G01N 33/68

(52) U.S. Cl. CPC C07K 14/70539; G01N 33/68
USPC 530/351; 530/395; 530/391.7; 435/69.3

(57) ABSTRACT

The production of soluble HLA class II molecules, as well as methods of using the soluble HLA class II molecules so produced, are described herein.

### CROSS REFERENCE TO RELATED APPLICATIONS

The present application is a continuation of U.S. Ser. No. 12/859,002, filed Aug. 18, 2010; which claims benefit under 35 U.S.C. 119(e) of provisional applications U.S. Ser. No. 61/234,937, filed Aug. 18, 2009; and U.S. Ser. No. 61/333,827, filed May 12, 2010. The entire contents of each of the above referenced patent applications are hereby expressly incorporated herein by reference.

### STATEMENT REGARDING FEDERALLY SPONSORED RESEARCH OR DEVELOPMENT

Not Applicable.

### BACKGROUND OF THE INVENTIVE CONCEPT(S)

1. Field of the Invention

The presently disclosed and claimed inventive concept(s) relates generally to a methodology of expression of soluble HLA class II proteins in mammalian cells as well as to methods of utilizing said soluble HLA class II proteins.

2. Description of the Background Art

Human cells express on their surface an incredibly large number of membrane-bound proteins, all of which display individual properties and physiological functions. From this large array of surface cell proteins, a number of clinical procedures require characterization of the human major histocompatibility complex (MHC) class I and II membrane-bound molecules. The human MHC class I and class II molecules are known as human leukocyte antigens, or HLA. The HLA class I and class II molecules are responsible for presenting peptide antigens to receptors located on the surface of T-lymphocytes, Natural Killer Cells (NK), and possibly other immune effector and regulatory cells. Display of peptide antigens on the MHC I and MHC II molecules are the basis for the recognition of “self vs. non-self” and the onset of important immune responses such as transplant rejection, graft-versus-host-disease, autoimmune disease, and healthy anti-viral and anti-bacterial immune responses.

HLA class I and class II molecules differ from person to person. Each person expresses a different complement of class I and class II on the surface of their cells. For transplant purposes it is important to determine which of the multiple HLA expressed on a cell are recognized by the antibodies of another individual. Anti-HLA antibodies can lead to hyperacute organ rejection. It is often difficult to determine which of many HLA are recognized by antibodies because sera can have antibodies to non-HLA proteins, multiple HLA molecules, and sera may crossreact among different HLA molecules. With many human proteins, many HLA proteins, antibodies to multiple human proteins, and antibodies crossreactive to various HLA proteins, it can be difficult when screening patients for organ transplantation to ascertain which of the many HLA in the population, and expressed on an organ to be transplanted, are recognized by antibodies. Antibodies to HLA proteins may also lead to problems during the transfusion of blood products, whereby antibodies in the blood of the blood donor may react with the HLA class I and class II antigens of the recipient of the blood product. Antibodies in the blood product that recognize the recipient's HLA may lead to transfusion related acute lung injury (TRAM.

Class I MHC molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptide antigens bound and presented by HLA class II are derived from extracellular foreign antigens, such as products of bacteria that multiply outside of cells, wherein such products include protein toxins secreted by the bacteria or any other bacterial protein to which the human immune system might respond in a protective manner. In this manner, class II molecules convey information regarding the existence of pathogens in extracellular spaces that are accessible to the cell displaying the class II molecule. HLA class II expressing cells then present peptide antigens derived from the extracellular antigen/bacteria to immune effector cells, including but not limited to, CD4⁺ helper T cells, thereby helping to eliminate such pathogens. The elimination of such pathogens is accomplished by both helping B cells make antibodies against microbes, as well as toxins produced by such microbes, and by activating macrophages to destroy ingested microbes.

HLA class I and class II molecules exhibit extensive polymorphism generated by systematic recombinatorial and point mutation events; as such, hundreds of different HLA types exist throughout the world's population, resulting in substantial immunologic diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, and because of their tremendous diversity, large quantities of individual HLA proteins are required in order to effectively study transplantation, autoimmunity disorders, and for vaccine development.

However, there has been no readily available source of individual MHC/HLA molecules. The quantities of HLA protein available have been small and typically consist of a mixture of different HLA molecules. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from human cells requires time-consuming and cumbersome purification methods in order to separate individual HLA class I or class II molecules away from other HLA proteins expressed by the cell, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of the results cannot directly distinguish between the different HLA molecules, and one cannot be certain that any particular HLA molecule is responsible for a given result. Therefore, prior to the present invention, a need existed in the art for a method of producing substantial quantities of individual HLA class II molecules so that they can be readily purified and isolated independent of other HLA class II molecules. Such individual HLA molecules, when provided in sufficient quantity and purity as described herein, provides a powerful tool for studying and measuring immune responses.

The fact that HLA class I contains three genes of interest: HLA-A, B, and C, while HLA class II contains multiple gene products as well including, DRA, DRB, DPA, DPB, DQA, and DQB, must be taken into consideration. To add to the complexity of the system, these proteins are polymorphic and are expressed in a heterozygous fashion, meaning that each cell expresses one molecule form from the mother and another from the father, leading to the expression of many different MHC molecules on each cell surface. Furthermore, antibodies have a difficult time discriminating among various class II; this complicates the serologic selection of cells that express large numbers of a particular HLA class II, thus complicating the purification of a given HLA class II protein.

Identification of HLA class II molecules on the cell surface by serological methods is difficult and complex. The cross reactivity between these molecules and the high background given by other surface-bound molecules that have similarities to the MHC system make this task unreliable.

Therefore, there exists a need in the art for improved methods of expressing individual HLA class II molecules. There exists a need for selecting for high expression of an individual HLA protein, and there exists a need for purifying a specific HLA protein without copurification of mixtures of HLA proteins. The presently disclosed and claimed inventive concept(s) overcome the disadvantages and defects of the prior art by providing a method of producing and purifying individual soluble HLA class II trimolecular complexes, as well as methods of use of said complexes in methods of antibody detection and epitope discovery.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 depicts the arrangement of the T-cell receptor and associated molecules. (a) Two disulfide-bonded chains of the T cell receptor which form a heterodimer. These recognize peptides associated with MHC molecules. (b) Four chains, collectively termed CD3, that associate with the T cell receptor dimer and participate in its transport to the surface of the cell. The CD3 complex together with the zeta chains, which form a homodimer, transduce the signal after antigen has bound.

FIG. 2 depicts the interface of the antigen presenting cell (APC) and the T-cell. (a) Specific peptides are presented by MHC class II and are bound by the T-cell receptor (TCR) which is associated with the CD3 complex. (b) Recognition by the TCR is transduced by the CD3 complex. The intracellular tails of the CD3 molecules contain a single conserved motif known as an immunoreceptor tyrosine-based activation motif or ITAM for short, which is essential for the signaling capacity of the TCR. Phosphorylation of the ITAM on CD3 renders the CD3 chain capable of binding an enzyme called ZAP70 (zeta associated protein), a kinase that is important in the signaling cascade of the T cell.

FIG. 3 is a schematic representation of a soluble HLA class II trimolecular complex produced in accordance with the presently disclosed and claimed inventive concept(s).

FIG. 4 is a schematic diagram of a method of producing the soluble HLA (sHLA) class II trimolecular complex (of FIG. 3) in accordance with the presently disclosed and claimed inventive concept(s).

FIG. 5 is a schematic diagram of sHLA class II trimolecular complex production in a hollow fiber bioreactor unit.

FIG. 6 graphically depicts the production of sHLA class II DRB1*0103 produced in transfected cells, demonstrating the ability to scale up production from a T175 flask to a hollow fiber bioreactor unit (CELL PHARM®).

FIG. 7 graphically demonstrates the ability of commercially available monoclonal antibodies (mAb) and patient sera to specifically detect the sHLA DRB1*0103 produced in FIG. 6.

FIG. 8 graphically depicts the ability to produce multiple different sHLA class II complexes from transfected cells in accordance with the presently disclosed and claimed inventive methods.

FIG. 9 graphically depicts production in a bioreactor of milligram quantities of sHLA class II over time.

FIG. 10 demonstrates quantification of sHLA class II DRB*0103/DRA*0101 (produced in FIG. 9) using electrospray mass spectroscopy.

FIG. 11 illustrates the molecular weight results and analysis of the proteins from FIG. 10 and using electrospray ionization TOF mass spectrometry.

FIG. 12 illustrates the desalting profile for soluble DRB1*0101 peptides. The arrow indicates the fractions collected and pooled for Edman degradation.

FIG. 13 illustrates the RP-HPLC elution profile of soluble DRB1*0101 peptides.

FIG. 14 graphically illustrates Edman data for each amino acid for each cycle of Edman degradation.

FIG. 15 graphically illustrates Edman data comparing picomoles of amino acid per cycle of Edman degradation.

FIG. 16 depicts a table of peptides identified by the methods of FIGS. 12-15. The peptides are designated by the sequence identifiers SEQ ID NOS:1-8, whereas the amino acid sequences of the source proteins for said sequences are designated by the sequence identifiers SEQ ID NOS:9-15.

FIG. 17 illustrates alignments of three of the peptides of FIG. 16 (SEQ ID NOS: 5, 4, and 3, respectively) with the common motif for DRB1*0101.

FIG. 18 graphically depicts coupling of soluble DRB1*1101 ZP HLA Class II molecule to a solid support and use thereof to facilitate removal of HLA Class II specific antibodies in an ELISA format. Panel A: a diagram of the consecutive absorption matrix ELISA performed for specific antibody removal. Panel B: absorbance and retentate values from 3 different HLA Class II specific mAb antibodies: L243, OL (One Lambda), and 2H11 were subjected to the consecutive absorbance matrix.

FIG. 19 graphically depicts that DRB1*1101-specific human sera was recognized by soluble DRB1*1101 in an ELISA format.

FIG. 20 graphically depicts that soluble DRB1*1101 can be coupled to SEPHAROSE® and used to absorb HLA Class II specific antibody, 9.3F10. Panel A: soluble DRB1*1101 was coupled to FastFlow SEPHAROSE® and packed into a gravity column. mAb 9.3F10, which has DR reactivity, was passed over the column and flow thru was collected as fractions. Then the mAb was eluted using DEA (diethanolamine) buffer, pH 11.3, was added to the column, and fractions were collected. Panel B: two separate ELISAs for total mouse IgG and human HLA were also performed on the Flow Thru and Eluate to detect specific antibodies versus HLA proteins that might have been eluted off the column.

FIG. 21 graphically depicts that antibodies contained in human sera specific for DRB1*1101 can be removed by a DRB1*1101 specific column. Donor #1 sera was passed over the DRB1*1101 SEPHAROSE® column, and two 2 ml fractions of flow thru were collected. To elute, DEA buffer pH 11.3, was added to the column, and two 2 ml fractions were collected. Panel A: a direct DRB1*1101 ELISA was performed to detect the amount of DRB1*1101 specific antibodies that were left in the flow thru and eluate. Panel B: a total human IgG sandwich ELISA was also performed to evaluate passage of total human IgG.

FIG. 22 graphically depicts that soluble DRB1*1101 coupled SEPHAROSE® is specific for DRB1*1101 and not other DR alleles. Donor #2 sera was passed over the same DRB1*1101 column in the same manner as FIG. 21, and two fractions of the flow thru and one fraction of the eluate were evaluated for multi-allele DR reactivity.

FIG. 23 depicts the nucleic acid (SEQ ID NO:16) and amino acid (SEQ ID NO:17) sequences of a DRA*0101 alpha chain-leucine zipper construct. The highlighted sequence encodes a linker that connects DRA1*0101 allele's sequence to the leucine zipper motif's sequence. The underlined sequence encodes the leucine zipper motif.

FIG. 24 depicts the nucleic acid (SEQ ID NO:18) and amino acid (SEQ ID NO:19) sequences of a DRB1*0401 beta chain-leucine zipper construct. The highlighted sequence encodes a linker that connects DRB1*0401 allele's sequence to the leucine zipper motif's sequence. The underlined sequence encodes the leucine zipper motif.

FIG. 25 depicts the nucleic acid (SEQ ID NO:20) and amino acid (SEQ ID NO:21) sequences of a DRB1*0103 beta chain-leucine zipper construct. The highlighted sequence encodes a linker that connects DRB1*0103 allele's sequence to the leucine zipper motif's sequence. The underlined sequence encodes the leucine zipper motif.

### DETAILED DESCRIPTION OF THE INVENTIVE CONCEPT(S)

Before explaining at least one embodiment of the presently disclosed and claimed inventive concept(s) in detail by way of exemplary drawings, experimentation, results, and laboratory procedures, it is to be understood that the presently disclosed and claimed inventive concept(s) is not limited in its application to the details of construction and the arrangement of the components set forth in the following description or illustrated in the drawings, experimentation and/or results. The presently disclosed and claimed inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context. The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of genomic, cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the major histocompatibility antigens found in humans. As used herein, “HLA” is the human form of “MHC”.

The terms “MHC class I light chain” and “MHC class I heavy chain” as used herein will be understood to refer to portions of the MHC class I molecule. Structurally, class I molecules are heterodimers comprised of two noncovalently bound polypeptide chains, a larger “heavy” chain (α) and a smaller “light” chain ((β-2-microglobulin or 32m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3^{rd} extracellular domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC class I light chain”, “β-2-microglobulin”, and “β2 m” may be used interchangeably herein. Association of the class I heavy and light chains is required for expression of class I molecules on cell membranes.

Like MHC class I molecules, class II molecules are also heterodimers, but in this case consist of two nearly homologous α and β chains, both of which are encoded in the MHC. The class II MHC molecules are membrane-bound glycoproteins, and both the α and β chains contain external domains, a transmembrane anchor segment, and a cytoplasmic segment. Each chain in a class II molecule contains two external domains: the 33-kDa α chain contains α₁ and α₂ external domains, while the 28-kDa β chain contains β₁ and β₂ external domains. The membrane-proximal α₂ and β₂ domains, like the membrane-proximal 3^{rd} extracellular domain of class I heavy chain molecules, bear sequence homology to the immunoglobulin-fold domain structure. The membrane-distal domain of a class II molecule is composed of the α₁ and β₁ domains, which form an antigen-binding cleft for processed peptide antigen. The peptides presented by class II molecules are derived from extracellular proteins (not cytosolic intracellular peptide antigens as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane. Because the peptide-binding groove of MHC class II molecules is open at both ends while the corresponding groove on class I molecules is closed at each end, the peptides presented by MHC class II molecules are longer, generally between 13 and 24 amino acid residues long. Like class I HLA, the peptides that bind to class II molecules often have internal conserved “motifs’, but unlike class I-binding peptides, they lack conserved motifs at the carboxyl-terminal end, since the open ended binding cleft allows a bound peptide to extend from both ends.

The term “trimolecular complex” as used herein will be understood to refer to the MHC heterodimer associated with a peptide. An “MHC class I trimolecular complex” or “HLA class I trimolecular complex” will be understood to include the class I heavy and light chains associated together and having a peptide displayed in an antigen binding groove thereof. The terms “MHC class II trimolecular complex” and “HLA class II trimolecular complex” will be understood to include the class II alpha and beta chains associated together and having a peptide displayed in an antigen binding groove thereof.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “biological sample” as used herein will be understood to include, but not be limited to, serum, tissue, blood, cerebrospinal fluid, tears, saliva, lymph, dialysis fluid, organ or tissue culture derived fluids, and fluids extracted from physiological tissues. The term “biological sample” as used herein will also be understood to include derivatives and fractions of such fluids, as well as combinations thereof. For example, the term “biological sample” will also be understood to include complex mixtures.

The term “HLA protein” as used herein will be understood to refer to any HLA molecule, complex thereof or fragment thereof that is capable of being expressed on a surface of a non-human cell. Examples of HLA proteins that may be utilized in accordance with the presently disclosed and claimed inventive concept(s) include, but are not limited to, an HLA class I trimolecular complex, an HLA class II trimolecular complex, an HLA class II α chain and an HLA class II β chain. Specific examples of HLA class II α and/or β proteins that may be utilized in accordance with the presently disclosed and claimed inventive concept(s) include, but are not limited to, those encoded at the following gene loci: HLA-DRA; HLA-DRB1; HLA-DRB3,4,5; HLA-DQA; HLA-DQB; HLA-DPA; and HLA-DPB.

The term “mammalian cell” as used herein will be understood to refer to any cell capable of expressing a recombinant HLA protein (as defined herein above) on a surface thereof. Therefore, any “mammalian cell” utilized in accordance with the presently disclosed and claimed inventive concept(s) must contain the necessary machinery and transport proteins required for expression of MHC/HLA proteins and/or MHC/HLA trimolecular complexes on a surface of such cell. “Mammalian cells” utilized in accordance with the presently disclosed and claimed inventive concept(s) must have (A) machinery for chaperoning and loading MHC/HLA proteins, such as class I and class II proteins; and (B) such machinery must be able to interact and work with human HLA proteins, such as class I and class II proteins. Not all cells express class II MHC protein; only professional immune cells such as but not limited to dendritic cells (DC), macrophages, B cells, and the like express class II proteins. Therefore, when it is desired to express HLA class II protein in a mammalian, non-human cell, such non-human cell must express class II MHC for that species and contain the appropriate machinery for interacting and working with both that species' class II MHC as well as human HLA class II. However, the presently disclosed and claimed inventive concept(s) also includes the use of cells of other lineages that have been induced to express class II MHC, such as but not limited to, cytokines, cells that have been subjected to mutagenesis, and the like.

The term “mammalian cell” as used herein refers to immortalized mammalian cell lines and does not include animals or primary cells. Examples of “mammalian cells” that may be utilized in accordance with the presently disclosed and claimed inventive concept(s) include, but are not limited to, human and mouse DC lines, macrophage lines, and B cell lines.

MHC (major histocompatibility complex) or HLA (Human leukocyte antigen) Class II molecules are found only on a few specialized cell types, including macrophages, dendritic cells and B cells, all of which are professional antigen-presenting cells (APCs). The peptides presented by class II molecules are derived from extracellular proteins (not cytosolic as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane.

Like MHC class I molecules, class II molecules are also heterodimers, but in this case consist of two homologous peptides, an α and β chain, both of which are encoded in the MHC. Class II molecules are composed of two polypeptide chains, both encoded by the D region. These polypeptides (alpha and beta) are about 230 and 240 amino acids long, respectively, and are glycosylated, giving molecular weights of about 33 kDa and 28 kDa. These polypeptides fold into two separate domains; alpha-1 and alpha-2 for the alpha polypeptide, and beta-1 and beta-2 for the beta polypeptide. Between the alpha-1 and beta-1 domains lies a region very similar to that seen on the class I molecule. This region, bounded by a beta-pleated sheet on the bottom and two alpha helices on the sides, is capable of binding (via non-covalent interactions) a small peptide. Because the antigen-binding groove of MHC class II molecules is open at both ends while the corresponding groove on class I molecules is closed at each end, the antigens presented by MHC class II molecules are longer, generally between 15 and 24 amino acid residues long. This small peptide is “presented” to a T-cell and defines the antigen “epitope” that the T-cell recognizes.

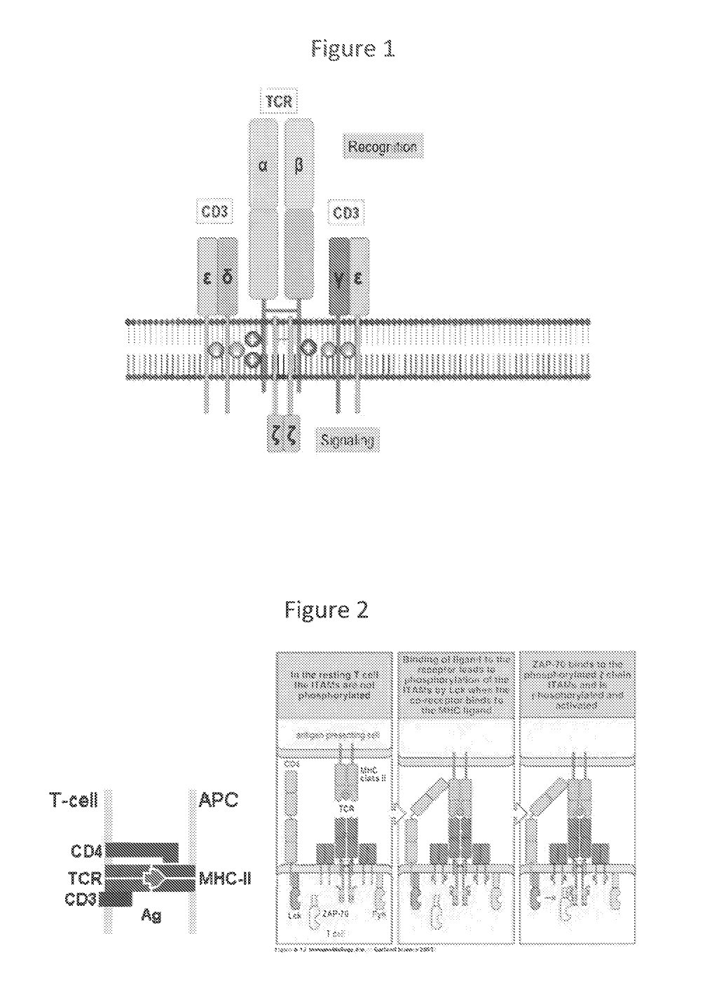

The T-cell receptor molecule (TCR) is structurally and functionally similar to the B-cell immunoglobulin receptor. TCR is composed of two, disulfide-linked polypeptide chains, alpha and beta, each having separate constant and variable domains much like immunoglobulins. The variable domain contains three hypervariable regions that are responsible for antigen recognition. Genetic diversity is ensured in a manner analogous to that for immunoglobulins. Thus, just like the B-cell surface immunoglobulin provides antigen specificity to its B-cell, the TCR allows T-cells to recognize their particular antigenic moiety. The diversity is shown in Table 1. However, T-cells cannot recognize antigen without help; the antigenic determinant must be presented by an appropriate (i.e., self) MHC molecule. Upon recognition of a specific antigen, the signal is passed to the CD3 molecule and then into the T-cell, prompting T-cell activation and the release of lymphokines. The following tables and FIGS. 1-2 illustrate the structure of the TCR as seen schematically.

The TCR provides the specificity for an individual T-cell to recognize its particular antigen. However, this recognition is “MHC-restricted” because the TCR also requires interactions with MHC. Also, interactions between the CD4 molecule (found on helper T-cells) and class II MHC or the CD8 molecule (found on cytotoxic T-cells) and class I MHC stabilize and consummate the antigen recognition process, allowing helper T-cells to respond to “exogenous” antigens (leading to B-cell activation and the production of antibody) or cytotoxic T-cells to respond to “endogenous” antigens (leading to target cell destruction). FIGS. 1 and 2 illustrate these processes schematically.

TABLE 1
Polymorphism of class II MHC genes

| | Number of alleles |
|---|---|
| Locus | (allotypes) |
| MHC-DPA | 12 |
| MHC-DPB | 88 |
| MHC-DQA | 17 |
| MHC-DQB | 42 |
| MHC-DRA | 2 |
| MHC-DRB1 | 269 |
| MHC-DRB3 | 30 |
| MHC-DRB4 | 7 |
| MHC-DRB5 | 12 |
| MHC-DM and MHC-DO | Relatively few alleles |

Binding of the MHC Class II molecule to the TCR has two primary effects. The first effect is Selectivity: different MHC molecules present different peptides to cells based on the biochemical rules of processing and peptide binding. Without presentation by MHC, an individual will not mount an immune response against cells that are expressing and presenting a given antigen. Therefore, there are immune dominant peptides that are chosen for presentation by particular MHC alleles for pathogen and self antigens. The second effect is Clonal Selection By stimulation of T-cells, MHC molecules can positively select or activate T-helper, T-regulatory or cytotoxic T-cell responses. However, these binding events can also tolerize particular T-cells by deletion or anergy. The later is due the reality that the interaction between the TCR and the MHC class II molecule provides specificity to a range of interactions that are necessary for T-cell activation. Binding of Therefore, manipulation of the T-cell response by provision of the MHC molecule in the context of a cell or in the absence of a cell B7.1 and CTLA-4 by CD28 and CD40 and CD40 ligand is necessary to augment CD3 signaling and induction of transcriptional changes in the CD4⁺ T-cell. These transcriptional activation events require modulation of NFkB and its activity to promote transcription of IL-2 which acts as an autocrine factor to amplify cell division and activation of the stimulated T-cell into an entire lineage sharing the specificity of the original cell. Further IL-2 stimulates CTL activities and secretion of IFN gamma for activation of further aspects of the adaptive immune response.

Based on the above, it is clear that MHC class II is central to a number of important events in the immune response cascade. Indeed, a large number of chronic inflammatory diseases and susceptibility to other diseases are associated with genes in the MHC class II region. For some diseases, this association is one of many; for others, it is the only confirmed association. Establishing the role for MHC class II unequivocally as the primary risk or catalyst of disease has been difficult due to many genetic factors and lack of precise disease models for chronic human disease. However, many genetic and functional immunological studies have associated particular alleles with specific diseases (Table 2).

TABLE 2
Disease-associated MHC class II molecules*

| | |
|---|---|
| Narcolepsy | |
| MHC-DQ6.1 MHC-DQA1*0102/DQB1*06011 | Negative |
| MHC-DQ6.2 MHC-DQA1*0102/DQB1*0602 | Positive |
| Coeliac disease | |
| MHC-DQ2 MHC-DQA1*0501/DQB1*0201 | Positive |
| MHC-DQ8 MHC-DQA1*0301/DQB1*0302 | Positive |
| Type 1 diabetes | |
| MHC-DQ2 MHC-DQA1*0501/DQB1*0201 | Positive |
| MHC-DR4.1 MHC-DRA1*0101/DRB1*0401 | Positive |
| MHC-DR4.3 MHC-DRA1*0101/DRB1*0403 | Negative |
| MHC-DR4.5 MHC-DRA1*0101/DRB1*0405 | Positive |
| MHC-DQ6 MHC-DQA1*0102/DQB1*0602 | Negative |
| MHC-DQ8 MHC-DQA1*0301/DQB1*0302 | Positive |
| Rheumatoid arthritis | |
| MHC-DR1 MHC-DRA1*0101/DRB1*0101 | Positive |
| MHC-DR4.1 MHC-DRA1*0101/DRB1*0401 | Positive |
| MHC-DR4.2 MHC-DRA1*0101/DRB1*0402 | Neutral or negative |
| Multiple sclerosis | |
| MHC-DR2a MHC-DRA5*0101/DRB5*0101 | Positive |
| MHC-DR2b MHC-DRA1*0101/DRB1*1501 | Positive |
| MHC-DQ6.2 MHC-DQA1*0102/DQB1*0602 | Positive |

*Positive association means that the associated MHC class II molecule increases susceptibility to the disease; negative association means that the associated MHC class II molecule protects against the disease; neutral association means that the associated MHC class II molecule has no effect on susceptibility to the disease (Jones et al., 2006).

As an example of the putative role of MHC class II as a catalyst for autoimmune dysfunction, diabetes is briefly discussed herein below. Diabetes risk and time to diabetes in relatives of patients directly correlates with the number of different autoantibodies present in the body. The pathogenesis of diabetes has been extensively studied, but the exact mechanism involved in the initiation and progression of beta cell destruction is still unclear. The presentation of beta cell-specific autoantigens by antigen-presenting cells (APC) [macrophages or dendritic cells (DC)] to CD4⁺ helper T cells in association with MHC class II molecules is considered to be the first step in the initiation of the disease process. Certain peptides and MHC class II molecule alleles are known to be highly correlative with diabetes and other autoimmune diseases (Todd et al., 1988; and Jones et al., 2006). Macrophages secrete interleukin (IL)-12 stimulating CD4⁺ T cells, to secrete interferon (IFN)-gamma and IL-2. IFN-γ stimulates other resting macrophages to release, in turn, other cytokines such as IL-1β, tumor necrosis factor (TNF)-α, and free radicals, which are toxic to pancreatic beta cells. During this process, cytokines induce the migration of beta-cell autoantigen specific CD8⁺ cytotoxic T cells. On recognizing specific autoantigen on β cells in association with class I molecules, these CD8⁺ cytotoxic T cells cause β cell damage by releasing perforin and granzyme and by Fas-mediated apoptosis of the beta cells. Continued destruction of beta cells eventually results in the onset of diabetes (Gronski and Weinem, 2006; Yoon and Jun, 2001; Yoshida and Kikutani, 2000; and Nepom and Kwok, 1998).

Due to the complex interplay required to activate a CD4⁺ T-cell, in addition to the TCR binding to the MHC class II, the decoupling of particular interactions from other spatially or temporally interactions will “short circuit” the cell this interaction can result in potent immune modulation. Researchers have explored use of a nonstimulatory anti-CD3 mAb (teplizumab) or anti-CTLA-4 mAb (ipilimumab) to be general down regulators of T-cell activities (Kaufman and Herold, 2009; Herold et al., 2009; and Weber et al., 2007). Encouraging results, including delayed progression, have been identified for each antibody in clinical development, however, since each have general effects to down regulate T-cell activities—including modulation of T-regulatory cells, other adverse events have been observed. Immune-related adverse events (IRAEs) have been observed in patients after CTLA-4 blockade and most likely reflect the drug mechanism of action and corresponding effects on the immune system. Early clinical data suggest a correlation between IRAEs and response to ipilimumab treatment. It would be expected that similar events will be noted with teplizumab as more patient experience accumulates. The IRAEs appear to arise from the rather general decoupling of the immune interaction. A more targeted decoupling associated directly with disease would be preferable.

Turning now to the presently disclosed and claimed inventive concept(s), the methods disclosed herein are directed to the expression of individual soluble HLA class II trimolecular complexes that are secreted from mammalian cells, and methods of isolating, purifying and/or using same. Such systems will have many advantages over the existing procedures of HLA serologic characterization.

The presently disclosed and claimed inventive concept(s) produces soluble HLA class II trimolecular complexes with advancements in the areas of purity, quantity, and applications over existing methods by using recombinant DNA methods to alter the protein in a manner that allows mammalian host cells to secrete the protein. HLA class II is naturally produced as a trimolecular complex that is endogenously loaded with peptide ligands and is bound to the membrane. Obtaining such naturally processed and loaded class II presently primarily proceeds by gathering membrane bound forms. Production of membrane bound class II requires cell populations to be lysed for capture of the complex. This method is known as cell lysate and represents state-of-the-art for natural mammalian HLA production for anti-HLA antibody detection assays. Cell lysate class II products are a mixture of numerous cell surface components, including the membrane anchored HLA class II trimolecular complex and other non-HLA proteins that decorate the cell membrane and that co-purify with HLA. Isolation of the HLA from other cell debris and membrane proteins reduces the yield of HLA class II. When producing HLA class II from detergent lysates, one is faced with either contaminating cell surface proteins and/or low class II protein yield. As an alternative, HLA class II can be obtained from Drosophila Schneider S-2 (insect) cell lines (Novak et al., 1999; and U.S. Pat. No. 7,094,555 issued to Kwok et al. on Aug. 22, 2006) and P. pastoris (yeast) (Kalandadze et al. 1996), whereby soluble forms of the HLA class II molecule have been produced. However, class II produced in insect cells lack the endogenously loaded peptides that are an integral component of the HLA class II native trimolecular complex. The HLA molecules produced in insect cells also lack the native glycosylation of mammalian cells. As insect cells lack mammalian protein glycosylation mechanisms and lack the chaperone complexes needed for natural peptide ligand loading, there is a reluctance to utilize class II proteins from insects for clinical applications.

The presently disclosed and claimed inventive concept(s) describes production of HLA class II by secretion from mammalian cells as a means to produce a native trimolecular complex free of contaminating membrane proteins. Through HLA class II secretion from mammalian cells, a pure product in which the predominant species is the desired HLA class II trimolecular complex is produced. A pure secreted molecule simplifies and enables downstream purification. Soluble HLA complexes are conducive to hollow fiber bioreactor production systems, such as but not limited to, the CELL PHARM® system (McMurtrey et al. 2008; Hickman et al., 2003; and Prilliman et al., 1999), as well as other systems designed for recombinant native protein secretion from mammalian cells. Highly concentrated harvests are much “cleaner” than cell lysates, thus allowing for minimal product loss because purification is simplified. Previously, only class I HLA has been secreted from mammalian cells in this manner; the presently disclosed and claimed inventive concept(s) is the first demonstration of successful secretion of HLA class II trimolecular complexes from mammalian cells.

Previously, HLA class II trimolecular complexes in native form have primarily been produced and purified via cell lysate methods; however, the complexes produced by these prior art methods have varying amounts of cell membrane secured to the purified HLA product, thereby creating several challenges for the yield of a homogeneous HLA product as well as problems associated with the use thereof. Quantification of the product produced by these methods is difficult, attachment of the prior art product to solid supports is complicated, and a precise understanding of the HLA trimolecular product is complicated by the copurification of a membrane and other proteins. The number of HLA trimolecular complexes per membrane piece is undefined with cell lysate methods. Molar equivalents of each different HLA class II complex are difficult to assess.

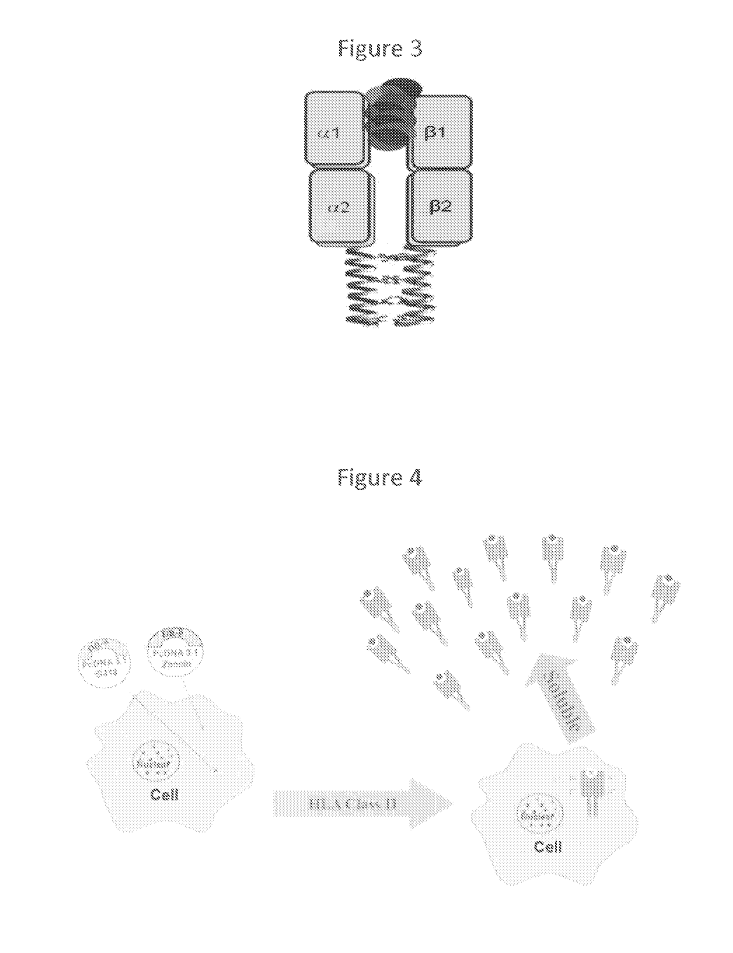

The presently disclosed and claimed inventive concept(s) is directed to a method to produce a soluble HLA class II trimolecular complex in mammalian cells that solves, in a unique and novel manner, the limitations seen when using cell lysate and insect cell techniques (FIG. 4 illustrates the method of production, while FIG. 3 represents the sHLA trimolecular complexes produced by said method). The presently disclosed and claimed inventive concept(s) overcomes the disadvantages and defects of the prior art through the use of a combination of methods; first, each of the α and β chains of the HLA class II complex is truncated such that the domain normally anchoring the complex to the cell surface is removed by recombinant DNA techniques. In native form, the alpha and beta chains of the HLA class II trimolecular complexes rely on the transmembrane domain to maintain a native conformation. While removal of this transmembrane domain facilitates secretion, this removal prevents formation of a trimolecular complex. The presently disclosed and claimed inventive concept(s) removes the transmembrane domain and replaces it with a super secondary structural motif, such as but not limited to, a leucine zipper protein sequence, which serves as a tethering moiety for the class II alpha and beta chains. The super secondary structural motif (such as but not limited to, a leucine zipper) thereby creates adhesion or fusion forces between proteins.

The unique combination of methods of the presently disclosed and claimed inventive concept(s) further includes the recombinant production of the soluble alpha and beta chains of the desired HLA class II in a mammalian cell line. The use of a recombinant mammalian cell line provides two distinct advantages over the prior art: first, production in a mammalian cell line allows the alpha and beta chains of the HLA class II molecule to be glycosylated in the same manner as seen for native HLA class II alpha and beta chains. Second, the mammalian cell line contains the appropriate machinery for natural endocytosis and lysosomal digestion to produce the same peptide ligands as would be produced by a native cell (referred to herein as an “endogenously produced peptide ligand”), as well as the appropriate chaperone machinery for trafficking and loading of the endogenously produced peptide ligands into an antigen binding groove formed between the alpha and beta chains of the HLA class II molecule.

Therefore, the features of (a) glycosylated, soluble HLA class II α and β chains; (b) production in a non-human mammalian cell line (or a human cell line that does not express endogenous class II molecules); and (c) a non-covalently attached, endogenously produced peptide ligand, provide distinct advantages that overcome the disadvantages and defects of the prior art cell lysate and non-mammalian cell production methods.

The development of an inexpensive way to produce soluble class II molecules presents an extraordinary opportunity for the diagnosis and treatment of transplantation, autoimmunity, infectious disease, and cancer. Such a targeted approach is offered through the soluble HLA class II complexes produced according to the presently disclosed and claimed inventive concept(s). Particular HLA class II complexes could be provided in the absence of the APC to engage the TCR without co-receptors and soluble immune stimulatory molecules. Decoupling of the APC and T-cell interaction is known to result in anergy or tolerance of lineage specific T-cell lines (Gronski and Weinem, 2006). This proposed mechanism is highly novel and enabled by the ability to produce authentic human, allele pure Class II antigen. When soluble HLA Class II-peptide complexes are multimerized, they can interact with the TCR on CD4⁺ T cells with high specificity and are useful for detection, isolation, elimination, activation, and/or inhibition of antigen specific T cells. There are reports of the requirement for specific peptides to be bound by these HLA class II antigens (Wicker et al., 1996), which could be used to induce tolerance or anergy—however, some interactions could be promoted without a specific peptide. Linkage of a particular peptide with a class II molecule could be mediated by both covalent and non-covalent methodologies. Further the HLA class II complex could be “monomeric” or “multi-meric” in structure; both forms may be applicable in diagnostic and/or therapeutic applications. Specific examples where this could be developed for diagnostics, highly specific reagents, and/or therapeutic purposes are presented below.

In one embodiment of the presently disclosed and claimed inventive concept(s), the soluble HLA class II trimolecular complex of the presently claimed and disclosed inventive concept(s) is produced by purifying the two associated HLA chains (i.e., alpha and beta chains for a single, specific allele) and be referred to as a monomer, due to the single nature of the complex represented.

In another embodiment, a composition comprising soluble HLA class II molecules of the presently claimed and disclosed inventive concept(s) is produced by multimerizing two or more soluble HLA class II trimolecular complexes. The term “multimer” as used herein will be understood to include two or more copies of the soluble HLA class II trimolecular complex which are covalently or non-covalently attached together, either directly or indirectly. The soluble HLA class II trimolecular complexes may be produced by any methods disclosed herein or by other methods disclosed in the art.

For multimerizing the two or more copies of the soluble HLA class II trimolecular complex, each of the HLA class II trimolecular complexes may be modified in some manner known in the art to enable attachment of the complexes to each other, or the multimer may be formed around a substrate to which each copy of the HLA class II trimolecular complex is attached. A tail may be attached to a portion of one or more of the two or more soluble HLA class II trimolecular complexes to aid in multimerization, wherein the tail may be selected from the group including but not limited to, a biotinylation signal peptide tail, an immunoglobulin heavy chain tail, a TNF tail, an IgM tail, leucine zipper, a Fos/Jun tail, and combinations thereof. The multimer can contain any desired number of HLA class II trimolecular complexes and thus form any multimer desired, such as but not limited to, a dimer, a trimer, a tetramer, a pentamer, a hexamer, and the like. Specific examples of multimers which may be utilized in accordance with the presently disclosed and claimed inventive concept(s) are described hereinbelow; however, these examples are not to be regarded as limiting, and other methods of multimerization known to those of skill in the art are also within the scope of the presently disclosed and claimed inventive concept(s). Streptavidin has four binding sites for biotin, so a BSP (biotinylation signal peptide) tail may be attached to a portion of the HLA molecule during production thereof, and a tetramer of the desired soluble HLA class II trimolecular complex could be formed by combining the trimolecular complexes with the BSP tails with biotin added enzymatically in vitro. An immunoglobulin heavy chain tail may be utilized as a substrate for forming a dimer, while a TNF tail may be utilized as a substrate for forming a trimer. An IgM tail could be utilized as a substrate for forming various combinations, such as tetramers, hexamers and pentamers. In addition, the soluble HLA class II trimolecular complexes may be multimerized through liposome encapsulation or artificial antigen presenting cell technology (see U.S. Ser. No. 10/050,231, filed by Hildebrand et al. on Jan. 16, 2002, the contents of which are hereby expressly incorporated herein by reference). Further, the soluble HLA class II trimolecular complexes may be multimerized through the use of polymerized streptavidin and would produce what is termed a “STREPTAMER®” (IBA GmbH, Gottingen, Germany).

The soluble HLA class II trimolecular complexes of the presently disclosed and claimed inventive concept(s) may further be modified for providing better performance and/or for aiding in stabilization of the monomer or multimer. Examples of modifications that may be utilized in accordance with the presently disclosed and claimed inventive concept(s) include but are not limited to, modifying an anchor and/or tail attached to the soluble HLA class II trimolecular complex as described herein above, modifying one or more amino acids in the peptide/HLA complex, PEGylation, chemical cross-linking, changes in pH or salt depending on the specific peptide of the soluble HLA class II trimolecular complex, addition of one or more chaperone proteins that stabilize certain soluble HLA class II trimolecular complexes, combinations thereof, and the like.

The presently disclosed and claimed inventive concept(s) represents the first endogenously loaded native HLA class II in soluble form. Endogenously loaded class II is a key element of the presently disclosed and claimed inventive concept(s) that separates it from the prior art. The endogenous peptide allows the class II trimolecular complex to be used in at least two specific applications not previously possible in soluble forms of the prior art (U.S. Pat. No. 7,094,555, previously incorporated herein by reference; Novak et al., 1999; and Kalandadze et al., 1996). First, only a HLA class II in its native trimolecular complex form can properly bind HLA class II specific antibodies. Similarly, the effects of a non-glycosylated HLA molecule on the conformation of class II antibody epitopes when used for HLA specific antibody detection or T-cell solicitation are unknown, but there is some evidence that improper glycosylation disrupts antigen presentation (Guerra et al., 1998). Therefore, the most advantageous format for HLA class II production is to maintain all components in a native form. It has been shown that HLA specific antibody recognition is impacted indirectly by the peptides that are part of the class I complexes (Wilson, 1981). The native binding of HLA specific antibodies is a key element of the presently disclosed and claimed inventive concept(s) when the sHLA described and claimed herein is used as the antigen in an HLA antibody sera screening assay. Another important application for native HLA loaded with endogenous peptides is to use the HLA class II for direct discovery of peptide epitopes that distinguish infected or autoimmune cells from “healthy” cells (McMurtrey et al., 2008; Hickman et al., 2003; and Prilliman et al., 1999). Prior art is limited to indirect epitope discovery, where soluble HLA dimers secreted from insect cell lines are loaded with synthesized peptides to be tested for T cell recognition (U.S. Pat. No. 7,094,555; and Novak et al., 1999). Indirect class II epitope discovery does not allow the detection of up regulation of self peptide presentation during infection.

One of the clear advantages of the direct class II epitope discovery approach made possible by the presently disclosed and claimed inventive concept(s) is that extracellular proteins are naturally endocytosed and digested in lysosomes for endogenous loading (McMurtrey et al., 2008; Hickman et al., 2003; and Prilliman et al., 1999). This process creates a natural sampling of numerous extracellular proteins for presentation in the trimolecular complex along with all the unique peptides that are presented during infections. Epitopes discovered by indirect methods could have unforeseen restrictions created by the complex chaperone system of mammalian cells that are deficient in insect cells and yeast. Additionally, the length of the synthetic epitopes determined by indirect methods may not correspond directly to the endogenously loaded peptide length.

A primary application of the secreted class II product described herein is the screening of patients awaiting a transplant for anti-HLA antibodies. The requirement for an anti-HLA antibody screening assay is based on the observation that particular events (such as but not limited to, blood transfusion, bacterial infection, and pregnancy) cause one individual to produce antibodies directed against the HLA of other people (Bohmig et al., 2000; Emonds et al., 2000; and Howden et al., 2000). Such anti-HLA antibodies must be detected before a patient receives a transplant, or the transplanted organ will be immediately rejected. Thus, screening for anti-HLA class II antibodies is a prerequisite for organ transplantation.

All transplant patients (approximately 20,000 a year in the U.S.) and all those waiting for a transplant (more than 60,000 a year in the U.S.) must regularly (monthly is preferred) be screened for antibodies that target the HLA of other people. The presently disclosed and claimed inventive concept(s) further includes methods of using the secreted or soluble HLA (sHLA) class II products described herein above; said products provide native proteins for quickly and accurately identifying anti-HLA antibodies in those awaiting a transplant. This pre-transplant diagnostic test will prevent rapid organ failure.

In addition to pre-transplant diagnostics, two post-transplant applications exist for the sHLA class II trimolecular complexes of the presently disclosed and claimed inventive concept(s). One application is a post-transplant diagnostic, whereby patients can be monitored for the production of antibodies to the HLA found on the transplanted organ (Muller-Steinhardt et al., 2000). A physician that finds their patient making antibodies to the HLA of the transplanted organ can increase immunosuppressive treatment. Sensitive and accurate antibody screening will indicate when a transplanted organ is being damaged. In a further embodiment, soluble HLA DR or other class II alleles could be used to develop diagnostic tests to detect anti-HLA-DR antibodies in recipients of organ transplants, bone marrow transplants, and—in the near future—stem cells transplants.

A second post-transplant and pre-transplant application is the removal of antibodies targeted to the transplanted organ. Soluble HLA class II complexes can be used to absorb out antibodies directed to the specific HLA molecule of the complex. Such antibody removal is useful when a patient attacks their transplanted organ with anti-HLA antibodies. Anti-HLA antibodies can also be removed prior to transplantation to enable better outcomes. The removal of antibodies specific for a particular HLA class II lessens the need for immune suppressing drugs. Precedence for this procedure exists in the removal of antibodies causing arthritis (Pratesi et al., 2000; and Schuna et al., 2000).

The presently disclosed and claimed inventive concept(s) therefore provides a method of producing individual, soluble HLA class II trimolecular complexes. In the method, a first isolated nucleic acid segment is provided, wherein the first isolated nucleic acid segment encodes a soluble form of an alpha chain of at least one HLA class II molecule, and a second isolated nucleic acid segment is provided, wherein the second isolated nucleic acid segment encodes a soluble form of a beta chain of the at least one HLA class II molecule. The isolated nucleic acid segments may be present in a single recombinant vector, or the isolated nucleic acid segments may be present on two separate recombinant vectors. The coding regions encoding the transmembrane domains of the alpha and beta chains have been removed and replaced with a super secondary structural motif that enables the alpha and beta chains (which previously interacted through their transmembrane domains) to interact. In one embodiment, the super secondary structural motif is a leucine zipper protein sequence that acts as a tethering moiety for the alpha and beta chains.

The isolated nucleic acid segments may be provided by any methods known in the art, including commercial production of synthetic segments. In one embodiment, the nucleic acid segments may be provided by a method that includes the steps of PCR amplification of the alpha and beta alleles from genomic DNA or cDNA. Methods of obtaining gDNA or cDNA for PCR amplification of MHC are described in detail in the inventor's earlier applications U.S. Ser. No. 10/022,066, filed Dec. 18, 2001 and published as US 2003/0166057 A1 on Sep. 4, 2003; and U.S. Pat. No. 7,521,202, issued Apr. 21, 2009; the entire contents of which are hereby expressly incorporated herein by reference. Therefore, while the following non-limiting example begins with gDNA and utilizes PCR amplification, it is to be understood that the scope of the presently disclosed and claimed inventive concept(s) is not to be construed as limited to any particular starting material or method of production, but rather includes any method of providing an isolated nucleic acid segment known in the art.

In one particular embodiment of the presently disclosed and claimed inventive concept(s), gDNA is obtained from a sample, wherein portions of the gDNA encode a desired individual HLA class II molecule's alpha chain and beta chain. Two PCR products are then produced: a first PCR product encoding a soluble form of the desired HLA class II alpha chain, and a second PCR product encoding a soluble form of the desired HLA class II beta chain. Each of the PCR products is produced by PCR amplification of the gDNA, wherein the amplifications utilize at least one locus-specific primer having a leucine sequence incorporated into a 3′ primer, thereby resulting in PCR products that do not encode the cytoplasmic and transmembrane domains of the desired HLA class II alpha or beta chains and thus produce PCR products that encode soluble HLA class II alpha or beta chains. The 3′ primer utilized for PCR amplification of the HLA class II alpha chain may incorporate the leucine sequence consistent with the acid sequence of the leucine zipper dimer, while the 3′ primer utilized for PCR amplification of the HLA class II beta chain may incorporate the leucine sequence consistent with the basic sequence of the leucine zipper dimer. However, it is to be understood that the description of the leucine zipper moiety is for purposes of example only, and that the presently disclosed and claimed inventive concept(s) encompasses the use of any super secondary structural motif that enables the alpha and beta chains (which previously interacted through their transmembrane domains) to interact.

One the isolated nucleic acid segments are provided, they are then inserted into at least one mammalian expression vector to form at least one plasmid containing the PCR products encoding the soluble HLA class II alpha chain and the soluble HLA class II beta chain. It is to be understood that the two nucleic acid segments may be inserted into the same vector or separate vectors.

The plasmid(s) containing the two PCR products are then inserted into at least one suitable immortalized, mammalian host cell line, wherein the cell line contains the necessary machinery and transport proteins required for expression of HLA proteins and/or are able to naturally process proteins into peptide ligands capable of being loaded into antigen binding grooves of HLA class II molecules.

The cell line is then cultured under conditions which allow for expression of the individual soluble HLA class II alpha and beta chains and production of functionally active, individual soluble HLA class II trimolecular complexes, wherein the soluble HLA class II trimolecular complexes comprise a soluble alpha chain, a soluble beta chain and an endogenously loaded peptide displayed in an antigen binding groove formed by the alpha and beta chains. The functionally active, soluble individual HLA class II trimolecular complex maintains the physical, functional and antigenic integrity of a native HLA trimolecular complex.

The presently disclosed and claimed inventive concept(s) also may be used to develop specific reagents and assays, including:

Class II sHLA Tetramers:

while tetramers of full length class II MHC are available, they are difficult to use and are very expensive. The presently disclosed and claimed inventive concept(s) provide a method to produce HLA class II molecules at a reasonable cost, and thus directly multiplied the applications of their use. For example, class II sHLA can be used in combination with libraries of overlapping peptides (from relevant self antigens, such as GAD in diabetes (DR3)), to identify and monitor the reactivity of auto-reactive CD4⁺ T cells (see below).

Defined Antigen/Pathogen/Tumor Antigen/Class II:

MHC Class I-peptide tetramers have been widely used to study human CD8⁺ T cell immune responses. There is a compelling need to extend this to MHC Class II and CD4⁺ T cells as well, which has historically been much more difficult to achieve. Multimerized sHLA Class II-peptide complexes created with defined pathogen or tumor antigens in their grooves provide useful reagents in a number of settings. First, these reagents may be used for testing vaccine efficacy. For some pathogens, CD4⁺ T cells are crucial for protection. In these situations, the availability of sHLA Class II-peptide tetramers enhances the development of vaccines by allowing monitoring of the relevant population. Even in classic viral infections where antibodies or CD8⁺ T cells are vital, CD4⁺ T cells orchestrate the response by producing cytokines for an optimal CD8⁺ T cell response or by providing help for the antibody response. For example, a CD4⁺ T cell response is required for optimal immunity to influenza. sHLA Class II-peptide multimers could be used to measure the efficacy of novel H1N1 vaccines, or in different groups of people (such as but not limited to, the elderly, pregnant, and the like).

A second use for these class II sHLA-peptide multimer reagents is in cancer immunotherapy monitoring. HLA Class I reagents have been developed to monitor the CTL response to various tumor antigens in a number of immunotherapy approaches (DC, adenovirus, gene gun, etc). While this is useful, it is critical to develop means to monitor the CD4⁺ response as well, particularly since this population contains regulatory T cells (T_{reg}) that can cause CTL effector dysfunction and thereby play a critical role in the success or failure of the therapy.

A third use for these class II sHLA-peptide multimer reagents is in chronic viral infection monitoring and/or treatment. While applicable to many viral diseases, hepatitis C virus is provided as a non-limiting example that is a highly persistent human pathogen that causes chronic liver disease. Impaired anti-viral effector mechanisms are associated with increased antigen specific CD4⁺T_{reg} cells. Thus, use of HCV peptide HLA Class II molecules (likely multimerized) is useful in monitoring the efficacy of various treatment options or in determining the best candidates for treatment.

Yet another use for these class II sHLA-peptide multimer reagents is in evaluating autoimmune disease progression. HLA Class II-peptide multimers may further be used in evaluating autoimmune diseases where the major human autoantigens are known. For example, the number of insulin specific CD4⁺ T cells may be used to monitor disease progression in T1 diabetes; the number of myelin specific CD4⁺ T cells could be used to predict relapses in diseases such as but not limited to, multiple sclerosis.

The challenges in this setting have been the high polymorphism of all HLA molecules and the relative difficulty of creating soluble HLA Class II reagents for each allele. Upon lowering these technical hurdles as with the presently disclosed and claimed inventive concept(s), it becomes possible to imagine the creation of large panels of HLA Class I and Class II peptide complexes suitable for screening use in a wide range of patients with a variety of diseases. The ability to readily produce high quantities of pure, human, mammalian-produced, glycosylated and folded HLA Class II at scale provides for these applications.

It has been difficult to identify binding peptides for a wide range of HLA Class II alleles and suballeles. Since soluble MHC Class II molecules have recently been used to identify binding peptides using microchip approaches (Gaseitsiwe and Maeurer, 2009), a robust new method to produce soluble HLA Class II molecules will be useful in discovering new allele specific peptide epitopes, thereby again facilitating the potential use of HLA-Class II peptide multimers for diagnostic purposes in the general population.

The presently disclosed and claimed inventive concept(s) is also related to methods of epitope discovery and comparative ligand mapping which can be utilized to distinguish diseased cells (i.e., infected or tumor cells) from non-diseased cells (i.e., uninfected or non-tumor cells) by unique epitopes presented by HLA molecules in the disease or non-disease state.

The presently disclosed and claimed inventive concept(s) is directed to a method for identifying at least one individual, endogenously loaded peptide ligand that distinguishes a diseased cell from a non-diseased cell. In the method, a non-diseased cell line containing a construct(s) as described herein above is provided. The construct(s) encodes: (1) an individual soluble HLA class II alpha chain and (2) an individual soluble HLA class II beta chain. In the same manner, a diseased cell line containing said construct(s) is also provided. The diseased cell line may be an infected cell line that has been infected with a microorganism, or the diseased cell line may be a tumorigenic cell line.

Both the non-diseased cell line and the diseased cell line are able to naturally process proteins into peptide ligands capable of being loaded into antigen binding grooves of HLA class II molecules. The non-diseased cell line and the diseased cell line are cultured under conditions which allow for expression of individual soluble HLA class II alpha and beta chains from the construct(s), such conditions also allowing for endogenous loading of a peptide ligand in the antigen binding groove formed by the individual soluble HLA class II alpha and beta chains to provide individual soluble HLA class II trimolecular complexes prior to secretion of the individual soluble HLA class II trimolecular complexes from the cell. The secreted individual soluble HLA class II trimolecular complexes having the endogenously loaded peptide ligands bound thereto are isolated from both the non-diseased cell line and the diseased cell line; the endogenously loaded peptide ligands are then separated from the individual soluble HLA class II alpha and beta chains from the non-diseased cell line, and the endogenously loaded peptide ligands are also separated from the individual soluble HLA class II alpha and beta chains from the diseased cell line. The endogenously loaded peptide ligands from the non-diseased cell line and the endogenously loaded peptide ligands from the diseased cell line are then isolated, and the endogenously loaded peptide ligands isolated from the diseased cell line are compared to the endogenously loaded peptide ligands isolated from the non-diseased cell line. At least one individual, endogenously loaded peptide ligand is identified that differs between the endogenously loaded peptide ligands isolated from the diseased cell line and the non-diseased cell line.

The method may further comprise the step of identifying a source protein from which the at least one individual, endogenously loaded peptide ligand is obtained.

The at least one endogenously loaded peptide ligand identified by the method described herein above may be obtained from a protein encoded by the non-diseased cell line. Said protein encoded by the non-diseased cell line from which the at least one endogenously loaded peptide ligand is obtained may have increased expression in a tumor cell line.

The step of identifying at least one individual, endogenously loaded peptide ligand may further be defined as identifying at least one individual, endogenously loaded peptide ligand presented by the individual soluble HLA class II molecule on the diseased cell line that is not presented by the individual soluble HLA class II molecule on the non-diseased cell line. In this manner, if the diseased cell line is an infected cell line, wherein the infected cell line has been infected with a microorganism, the at least one endogenously loaded peptide ligand so identified may be obtained from a protein encoded by the microorganism with which the cell line was infected.

Alternatively, the step of identifying at least one individual, endogenously loaded peptide ligand may further be defined as identifying at least one individual, endogenously loaded peptide ligand presented by the individual soluble HLA class II molecule on the non-diseased cell line that is not presented by the individual soluble HLA class II molecule on the diseased cell line.

The presently disclosed and claimed inventive concept(s) also includes a method in which a substrate is provided, wherein the substrate is selected from the group consisting of a well, a bead (such as but not limited to, flow cytometry bead and/or a magnetic bead), a membrane (such as but not limited to, a nitrocellulose membrane, a PVDF membrane, a nylon membrane, and acetate derivative), a microtiter plate, a matrix, a pore, plastic, glass, a polymer, a polysaccharide, nylon, nitrocellulose, a paramagnetic compound, and combinations thereof. Next, a functionally active, soluble individual HLA class II trimolecular complex purified substantially away from other proteins such that the soluble, individual HLA class II trimolecular complex maintains the physical, functional and antigenic integrity of a native HLA class II trimolecular complex is provided. The functionally active, soluble individual HLA class II trimolecular complex may be purified as described above or by any other method known in the art. The functionally active, soluble individual HLA class II trimolecular complex comprises soluble alpha and beta chains with an endogenously loaded peptide displayed in an antigen binding groove formed by the soluble alpha and beta chains. The functionally active, soluble individual HLA class II trimolecular complex is then directly or indirectly linked to the substrate, wherein the conformation of the functionally active, individual HLA class II trimolecular complex is maintained when the functionally active, individual HLA class II trimolecular complex is linked to the substrate.

The functionally active, soluble individual HLA class II trimolecular complex may be directly attached to the substrate, or the soluble HLA class II trimolecular complex may be indirectly attached to the substrate via an anchoring moiety. The anchoring moiety may be any moiety capable of attaching the HLA class II trimolecular complex to the substrate, including but not by way of limitation, an anti-HLA antibody (such as but not limited to, any of the anti-HLA class II antibodies described herein) and a tail or tag (such as but not limited to, a histidine tag, a biotinylation signal peptide, a VLDLr tail or a FLAG tail).

The presently disclosed and claimed inventive concept(s) is also directed to a method of detecting and/or removing anti-HLA antibodies in a biological sample utilizing the sHLA trimolecular complexes described herein above. Such method includes providing a substrate having functionally active, soluble individual HLA class II trimolecular complexes attached thereto, as described herein above. A biological sample is then reacted with the substrate having the functionally active, individual HLA class II trimolecular complex linked thereto, and the substrate is washed to remove unbound portions of the biological sample. The substrate having the functionally active, individual HLA class II trimolecular complex linked thereto is then reacted with means for detecting anti-HLA antibodies, and it is determined that anti-HLA antibodies specific for the individual HLA class II trimolecular complex are present in the biological sample if the means for detecting anti-HLA antibodies is positive.

The presently disclosed and claimed inventive concept(s) also includes a method for removing anti-HLA antibodies from a biological sample. The method includes providing a substrate having functionally active, soluble individual HLA class II trimolecular complex attached thereto as described herein above. A biological sample is then reacted with the substrate having the functionally active, individual HLA class II trimolecular complex linked thereto, whereby antibodies specific for the HLA class II trimolecular complex are removed from the biological sample.

In a further embodiment, soluble HLA class II offers potential therapeutic applications due to its critical interactions with the TCR in initiating the immune response cascade. These applications include (but are not limited to):

Elimination.

HLA Class II-peptide multimers can be conjugated to ricin, strontium, selenium or other toxins and used to deplete unwanted antigen specificities in vivo. This could potentially be useful in the therapy of autoimmune diseases, again where the major target antigens are known (insulin in T1 diabetes, gliaden in celiac disease, desmoglein 3 in pemphigus, etc.)

Activation and Inhibition In Vivo:

HLA-peptide multimers have been shown to either activate or inhibit antigen specific immune responses in vivo in animal models. It may be useful to activate antigen specific immune responses in vivo to prime T cells prior to vaccination, particularly in tumor immunotherapy. It may be useful to inhibit undesirable immune responses with HLA Class II-peptide multimers in the autoimmune disease setting, such as type 1 diabetes, an application that has significant impact in the art.

Whether multimers activate or inhibit immune responses is dependent on the spatial constraints and multivalency of the reagent. For example, HLA Class I or Class II peptide monomers can be dimerized with Ig and tetramerized with streptavidin. Even higher order complexes can be created with lipid vesicles, nanoparticles, or fixed staph A particles. Also, the effect on immune responses in vivo can differ depending on whether a single injection is given (activation) or multiple injections are given (inhibition).

Cell Therapy:

HLA Class II-peptide multimers could be used to physically isolate antigen specific cells. This could be useful for cellular therapy, such as in the immunotherapy of melanoma or chronic infection with HCV or for CMV following stem cell transplant. An exciting variation on this would be to use HLA Class II-peptide multimers to isolate and expand antigen specific regulatory T cells for cell therapy in graft versus host disease and autoimmunity.

The presently disclosed and claimed inventive concept(s) further describes and claims soluble HLA class II molecules that offer specific treatment for a range of diseases, including:

Celiac Disease.

In this disease gliadin is translocated into epithelial cells of the intestinal mucosa. In these cells gliadin is then transported into MHC-DR rich compartments, and from there peptides are presented to CD4⁺ T cells. The key peptides are well known. In this case one could envision a topical (intestinal delivery system) of Class II molecules loaded with a low affinity peptide. Once inside the cells excess Class II molecules could exchange their peptide for the immunodominant pathogenetic peptide inducing anergy. A second approach could be to pre load the Class II molecule with a low affinity analog of the immunodominant gliadin peptide to induce anergy. A final possibility would be to use toxin-conjugated MHC-DR-peptide to eliminate disease causing gliadin-specific CD4⁺ T cells. The availability of soluble Class II molecules provides for multiple therapeutics.

Sepsis Induced by Trauma.

It is well known that septic patients immediately after trauma undergo a rapid loss of HLA DR on cells and in circulation. In sepsis due to Gram-positive bacteria there is a role for superantigens, which drive the lethal event associated with sepsis. It has been proposed that soluble HLA DR molecules can bind superantigen by complex formation functioning as an immunoadsorbent, hence preventing activation of T cells (i.e., in the Emergency Room where a bolus of HLA DR molecules is administered to patients with trauma sepsis and thus modulate morbidity and mortality).

Bone Marrow Transplant/Hematopoietic Stem Cell Transplants.

The embodiment explained below is applicable to both.

T cell reactivity in the form of graft vs. host disease (GVHD) is a common occurrence in bone marrow transplantation/hematopoietic stem cell transplants. One possible use of soluble HLA DR molecules would be to tolerize donor cells to the recipient Class II molecules by incubation of the donor transplant with soluble HLADR molecules. This will have to be sufficiently specific to avoid side effects. It could be used in combination with conventional pharmacological treatments of the recipient. This therapy could maximize the chances to control the occurrence of GVHD. Alternatively, flow cytometry with HLA class II multimers could be used to remove recipient specific T cells prior to infusion.

Organ Transplant.

In this case a recipient of a kidney or liver from a living donor could be pre-treated with the HLA DR molecules of the prospective donor to tolerize the recipient's T cells and avoid organ rejection. This provides a protective measure against acute (not hyperacute or chronic) rejection.

Corneal Transplant.

Induction of anterior chamber associated immune deviation (ACAID) shows that the anterior chamber of the eye is a site where it is relatively easy to initiate immune regulation. Immune deviation of course but also anergy induction is possible. For reasons we do not understand this can act at the systemic level, but there is not much reagent necessary to retain the ability to immune modulate or anergize.

Antigen Specific Anergy by Interfering with Peptide Loading and Presentation to T Cells.

This embodiment provides a general form of intervention in all those diseases in which a precise role for CD4⁺ T cells in pathogenesis has been established. This requires action inside the cell. This may be achieved by creating larger units by conjugating HLA DR molecules with (for example) transferrin. This complex is then very rapidly internalized by the transferrin receptor and the process would deliver the exogenous Class II molecules to the late endosomal compartment. If the excess Class II molecule is preloaded with an antagonist peptide, then one may switch the balance from activation to tolerance. In other words, the cells will present the agonist immunodominant and present the pathogenetic peptide much less.

T cell specific anergy may also be induced by providing soluble HLA-Class II loaded with disease specific peptides to target disease causing T cells. These soluble HLA molecules would bind the T cell receptors for the disease specific T cells but would not have the necessary costimulatory molecules to stimulate the T cell, switching the balance from activation to tolerance (anergy), by the “signal 1 without signal 2” hypothesis.

Immunogens for Cancer.

Tetramers loaded with specific tumor associated Class II peptides (e.g., p53, telomerase, MUC.1, NY-SO) may be used as a vaccine in cancer patients to induce antibodies (TCR antibodies)

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### Example 1

### Production of Class II sHLA Trimolecular Complexes

This Example is directed to the expression of soluble individual human HLA class II trimolecular complexes in mammalian immortal cell lines. The method includes the use of modifications that alter the endogenous membrane bound complexes in such a way that the membrane bound anchor is disrupted, thereby allowing the cell to secrete the HLA class II trimolecular complexes. In this Example, the Alpha and Beta chain genes encoding HLA class II-DR, HLA-DQ, and HLA-DP were truncated such that the transmembrane and cytoplasmic domains were deleted. At the site of the truncation, a leucine zipper (a tethering moiety) replaced the transmembrane and cytoplasmic that endogenously anchors HLA to the membrane. The leucine zipper allows the HLA to be secreted from the cell while maintaining the class II trimolecular complex native confirmation (FIGS. 3 and 4). The leucine zipper is comprised of an acid segment tailing the class II alpha chain with complementary basic domain tailing the class II beta chain. The acid and basic segments fuse by means of the amino acid leucine being placed every 7 amino acids in the d position of the heptad repeat. The strategy was used by Chang in 1994 to bind the alpha and beta chains of soluble T cell Receptors together in the same fashion.

HLA class II complexes are comprised of two different polypeptide chains, designated α and β. In one method, the alpha and beta constructs were commercially purchased and directly ligated into a mammalian expression vector. In another, the constructs were produced by PCT amplification as described in the paragraph below, followed by purification and ligation into a mammalian expression vector.

Amplification of specific HLA class II genes from genomic DNA or cDNA was accomplished using PCR oligonucleotide primers for alleles at the HLA-DRα HLA-DRA), DRβ (HLA-DRB); DQα (DQA), DQβ (DQB); or DPα (DPA) and DPβ (DPB) gene loci. The beta chain 3′ PCR primer incorporates the leucine sequence consistent with the basic sequence of the leucine zipper dimer. The Alpha chain 3′ primer incorporates the leucine sequence consistent with the acid sequence of the leucine zipper dimer. The truncation of the class II genes through placement of the PCR primers eliminates the cytoplasmic and transmembrane regions, thus resulting in a soluble form of HLA class II trimolecular complex with a leucine zipper moiety.

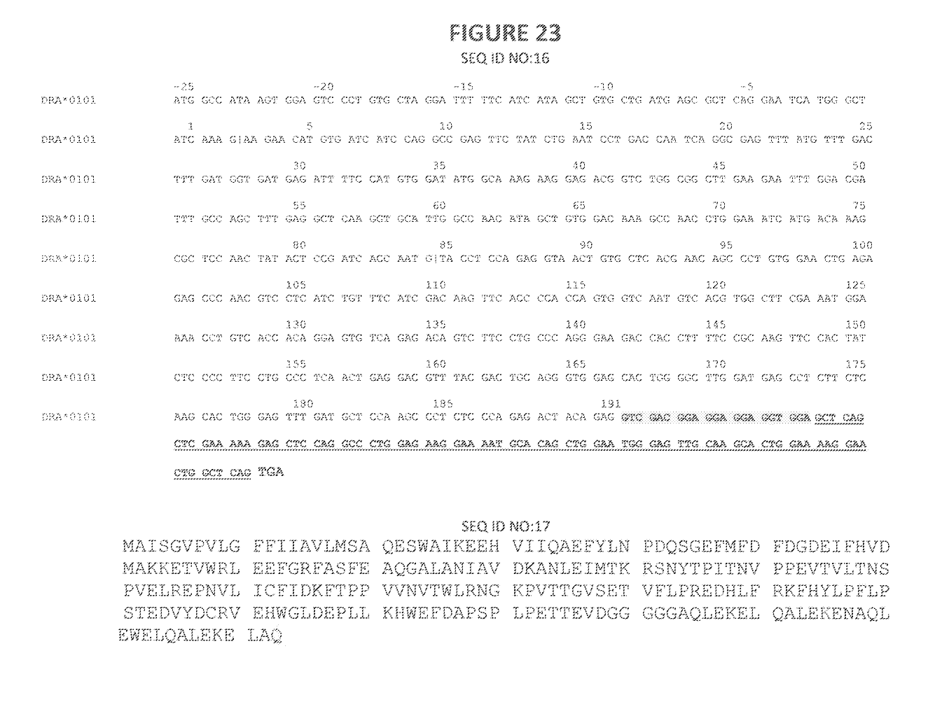

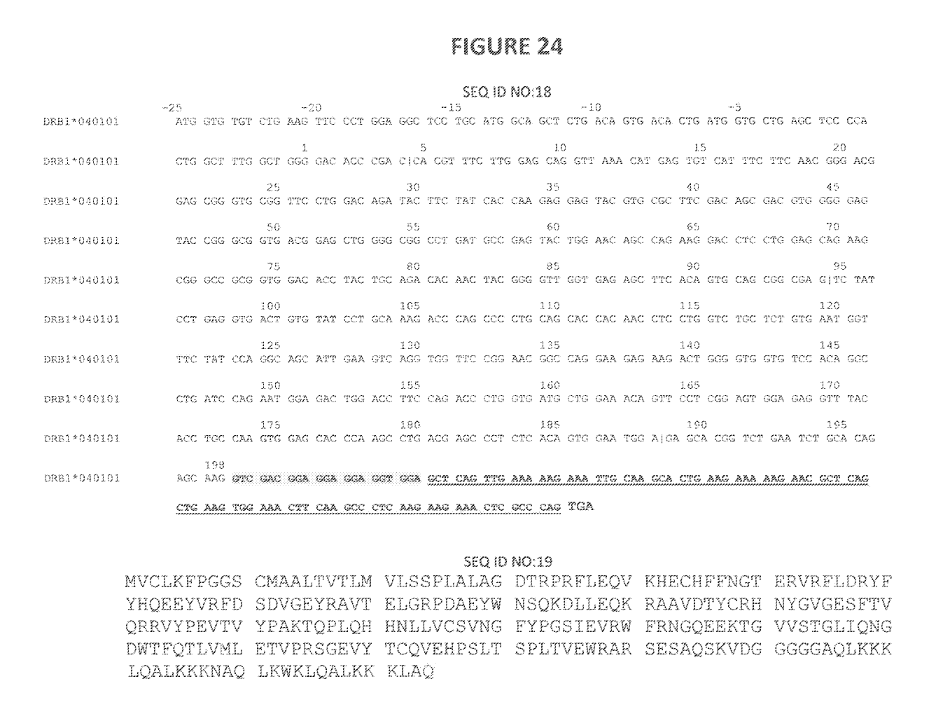

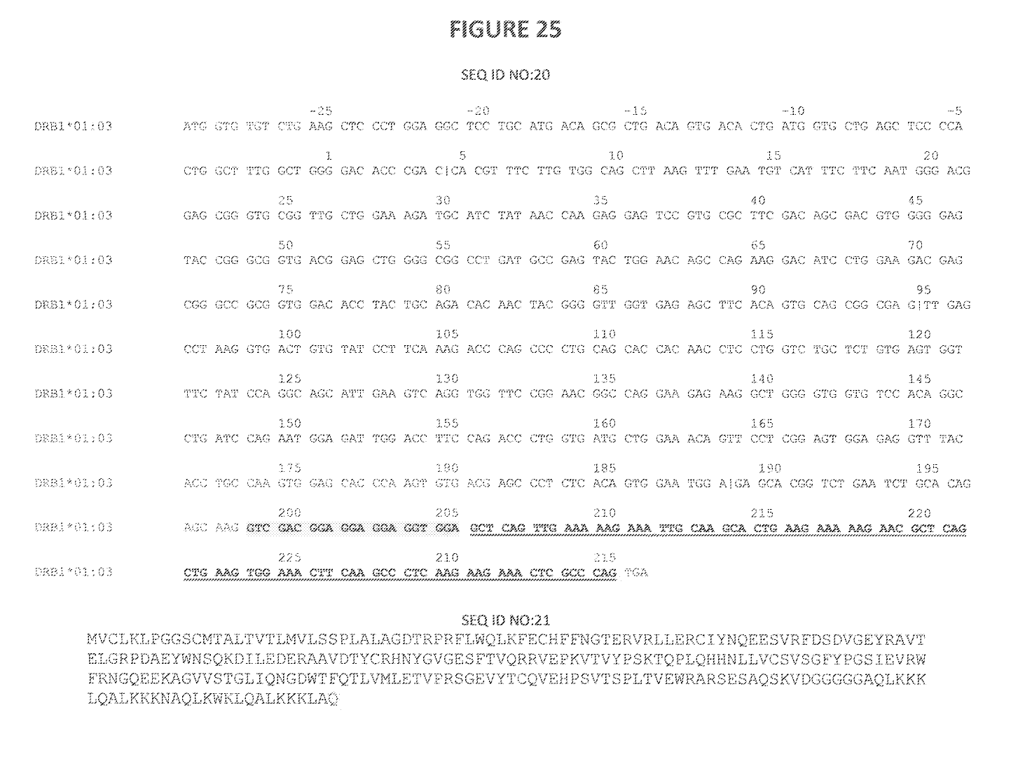

FIGS. 23-25 represent constructs used in the methods of sHLA production of the presently disclosed and claimed inventive concept(s). FIG. 23 illustrates the nucleic acid and amino acid sequences for a DRA1*0101 alpha chain-leucine zipper construct (SEQ ID NOS:16 and 17, respectively). FIG. 24 illustrates the nucleic acid and amino acid sequences for a DRB1*0401 beta chain-leucine zipper construct (SEQ ID NOS:18 and 19, respectively). FIG. 25 illustrates the nucleic acid and amino acid sequences for a DRB1*0103 beta chain-leucine zipper construct (SEQ ID NOS:20 and 21, respectively).

The constructs were then inserted into a mammalian expression vector. In one instance, the alpha chain was cut with one set of restriction enzymes, while the beta chain was cut with another set of restriction enzymes. The purified and cut alpha chain amplification products were ligated into the mammalian expression vector pcDNA3.1. Next, this ligated vector containing the sHLA class II alpha gene was transformed into E. coli strain JM109. The bacteria were grown on a solid medium containing an antibiotic to select for positive clones. Colonies from this plate were picked, grown and checked to contain insert. Plasmid DNA was isolated from the identified positive clones and subsequently DNA sequenced to insure the fidelity of the cloned alpha gene.

The alpha vector was re-cut using a second set of restriction enzymes which facilitate directional cloning of the purified beta PCR product. The final ligation product consisted of both alpha and beta clones. Plasmid DNA was then isolated from positive clones, and the beta genes were DNA sequenced from these clones.

Plasmid DNA for the alpha and beta class II alleles was prepared and DNA sequenced to confirm fidelity of the amplified class II genes. Log phase mammalian cells and the plasmid DNA were mixed in a plastic electrocuvette. This mixture was electroporated, placed on ice and resuspended in media. Special optimization was required for the electroporation step to enable successful enablement of the presently disclosed and claimed inventive concept(s). Standard electroporation procedures were unsuccessful in extensive trials by the inventors and as reported by other labs in publications.

After incubation for 2 days at 37° C. in a CO₂ incubator, the cells were subjected to selection with the antibiotic. First cells were counted and viability was determined. The cells were then resuspended in conditioned complete media. Next, cells were placed into each well of a 24-well plate and left to undergo selection. Supernatant from each well was taken, and an ELISA assay was performed to determine sHLA class II production. High producers were expanded and cryopreserved for large-scale production.

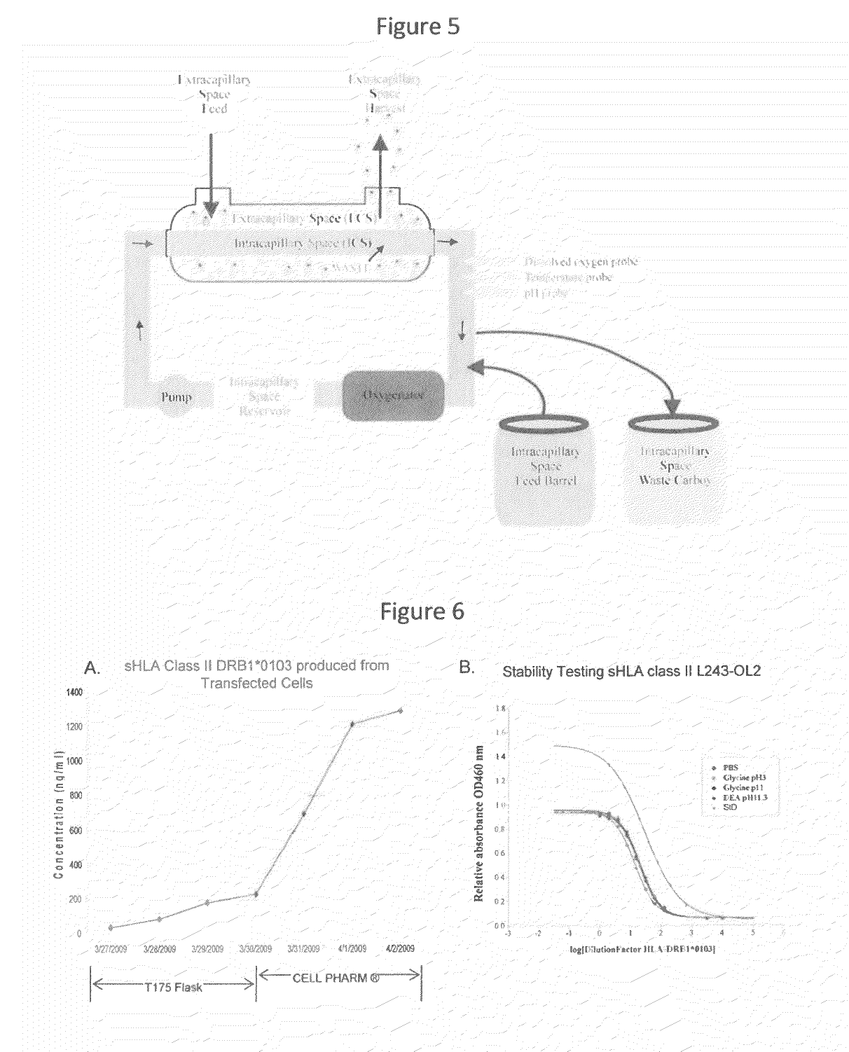

Prior to culture in CELL PHARM® bioreactors, the cellular growth parameters (pH, glucose, and serum supplementation) for each line was optimized for growth in bioreactors. Approximately 8 liters of naïve or pathogen infected sHLA-secreting class II transfectants were cultured in roller bottles in culture media supplemented with penicillin/streptomycin and serum or ITS (insulin-transferrin-selenium) supplement. The total volume of cells cultured was adjusted such that approximately 5×10⁹ cells were obtained. Cells were pelleted by centrifugation and resuspended in 300 ml of conditioned medium in a CELL PHARM® feed bottle. Cells and conditioned medium were inoculated through the ECS feed pump of a Unisyn CP2500 CELL PHARM® into 30 kDa molecular-weight cut-off hollow-fiber bioreactors previously primed with media supplemented with penicillin/streptomycin and serum or ITS. The culture of cells inside the CELL PHARM® was continued with constant monitoring of glucose, pH and infection. Medium feed rates were monitored and adjusted to maintain a glucose level of 70-110 mg/dL. FIG. 5 provides an overview of the cell pharm bioreactor system; the sHLA secreting cells and their sHLA product were contained within the extra capillary space (ECS) of the hollow fiber bioreactor. Nutrients and gases for the cells were provided by recirculated medium.

FIG. 6A illustrates the increased production of sHLA class II DRB1*0103 produced from transfected cells when scaled up to the bioreactor production. The sHLA was purified from the cell supernatant with the specific anti-HLA class II antibody L243 coupled to CNBr-activated SEPHAROSE® 4B, and the protein concentration determined by a micro-BCA protein assay, UV absorbance and ELISA. The sHLA class II titer of a typical production run was found to be approximately 4-5 mg/liter of growth media. FIG. 6B illustrates that these trimolecular complexes were very stable in a wide variety of buffers and at a wide range of pH concentrations using monoclonal antibody L243, which reacts with virtually all DR HLA proteins. L243 is a murine IgG2a anti-HLA-DR monoclonal antibody previously described by Lampson & Levy (J. Immunol. (1980) 125 293); said monoclonal antibody has been deposited at the American Type Culture Collection, 10801 University Boulevard, Manassas, Va. 20110-2209, under Accession number ATCC HB55.

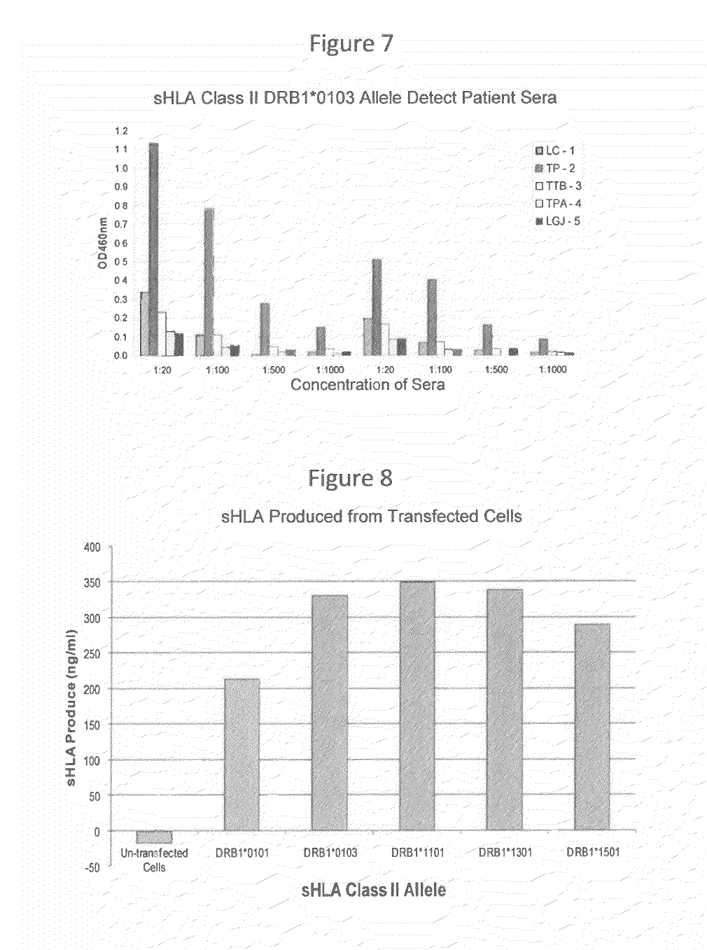

In FIG. 7, the serologic integrity of the purified sHLA class II trimolecular complexes was confirmed by directly coating the complexes on a plate and exposing the coated complexes to defined commercially available mAbs and patient sera. In addition, comparison of the sHLA with full-length molecules showed no differences in antigenicity.

FIG. 8 illustrates the ability to produce multiple different sHLA class II trimolecular complexes by the methods of the presently disclosed and claimed inventive concept(s). While DRB1*0101, DRB1*0103, DRB1*1101, DRB1*1301 and DRB1*1501 are shown for the purposes of example, multiple other sHLA class II trimolecular complexes have also been produced in milligram quantities in accordance with the presently disclosed and claimed inventive concept(s). Trimolecular complexes from each sHLA DR protein have been detected and quantitated using the L243 ELISA-based assay.

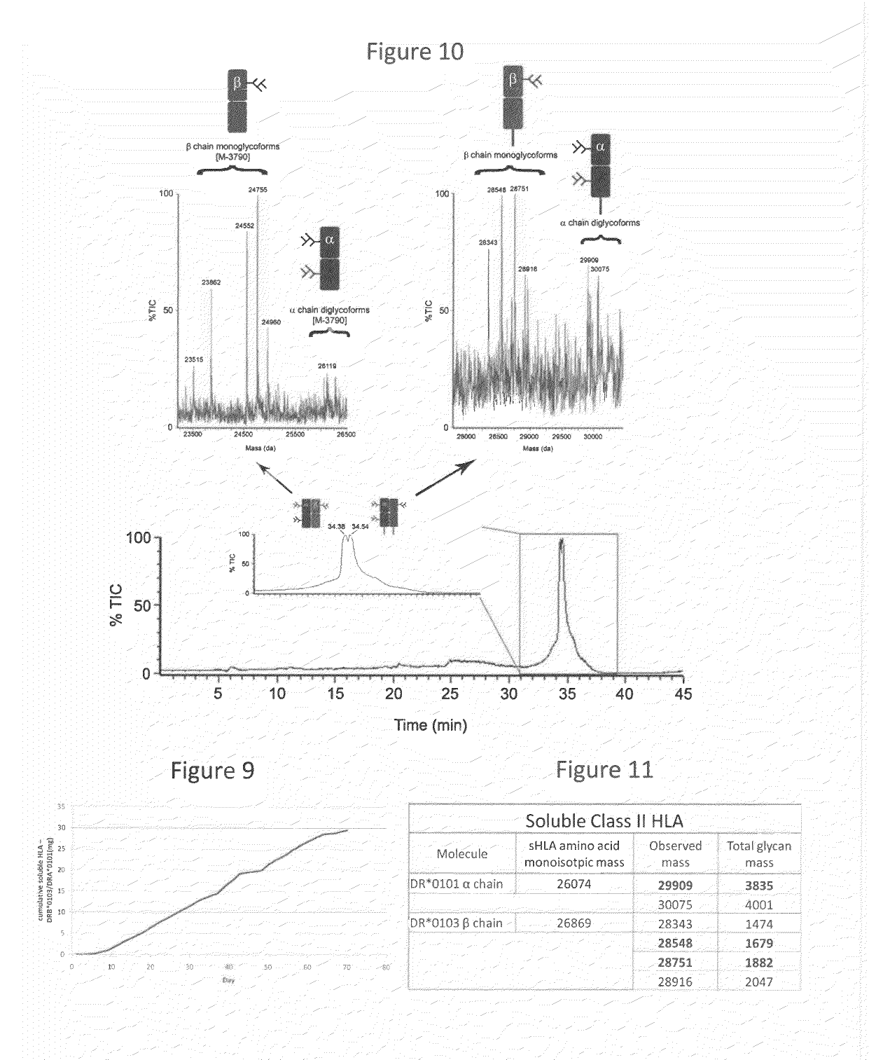

FIGS. 9-11 illustrate another example of sHLA class II production in accordance with the presently disclosed and claimed inventive concept(s). In this example, immortalized cells tranfected with a soluble HLA-DRB*0103/DRA*0101 construct (DRB1*0101 soluble alpha chain with leucine zipper and DRB1*0103 soluble beta chain with leucine zipper) were grown in a roller bottle format until a total 1¹⁰ cells were obtained. The cells were then seeded into the ECS portion of 2 hollow fiber bioreactor units. Cells were grown in DMEM+10% FBS in the ECS and no FBS in the ICS. ECS harvest was collected every day until cells were dead and no longer producing soluble HLA. Protein was quantified using a capture ELISA. For this ELISA an antibody specific for the leucine zipper (2H11) was used as the capture antibody, and an antibody specific for class II HLA (L243) as the detector antibody. Approximately 8 mg of soluble HLA was loaded on an affinity antibody (L243) column and eluted in an alkaline buffer (0.1M Glycine, pH 11). Fractions containing soluble HLA were pooled and lyophilized. The lyophilate was resuspended in water/20% acetonitrile and loaded onto a C18 RP-HPLC column. The soluble HLA was then eluted using a 20% to 80% acetonitrile gradient and detected using electrospray ionization TOF mass spectrometry.

As can be seen in FIG. 9, milligram quantities of a soluble form of a single class II HLA heterodimer were produced in the bioreactor format. Additionally, the intact heterodimer was purified with no other contaminating proteins, as determined by LCMS (FIG. 11). This soluble class II contains a monoglycosylated beta chain and diglycosylated alpha consistent with native class II HLA (FIG. 10). Furthermore, the various glycoforms were consistent with the natural variation in sugars that occurs as a protein transits to the cell surface. For a subpopulation of the class II molecules, intracellular proteolytic events removed all but two amino acids of the leucine zipper domain from both the alpha and the beta chains. However, like the full length construct, class II without the leucine zipper domain remain as a heterodimer as both the alpha and beta chains co-elute. These soluble class I and class II HLA proteins are amenable to analysis by mass spectrometry, whereby the purity and identity of these proteins can be confirmed by TOF analysis of molecular weights (FIG. 11).

### Example 2

### Purification of Class II sHLA and Analysis of Peptides Loaded Therein

Class II sHLA trimolecular complexes produced according to Example 1 were affinity purified using a monoclonal antibody against HLA-DRB. L243 is a murine IgG2a anti-HLA-DR monoclonal antibody previously described by Lampson & Levy (J. Immunol. (1980) 125 293); said monoclonal antibody has been deposited at the American Type Culture Collection, 10801 University Boulevard, Manassas, Va. 20110-2209, under Accession number ATCC HB55. A 50 mL L243 affinity column was prepared by coupling said antibody to CNBr-SEPHAROSE® 4B Fast Flow resin.

Soluble DRB1*0101 trimolecular complexes were then purified by passing approximately 30 L of CELL PHARM® supernatant over the 50 mL L243 column. The flow through was checked periodically by ELISA for unbound DRB1*0101. None was found, so the column was binding all of the HLA class II.

Once all the supernatant was loaded, the column was washed with 1600 mL of 20 mM sodium phosphate, pH 7.2. The column was moved to the AKTA™ purification system (GE Healthcare Biosciences Corp, Piscataway, N.J.) for elution of the class II sHLA trimolecular complexes. The class II sHLA trimolecular complexes were eluted with 50 mM diethylamine (DEA), pH 11.3 into a lyophilizing jar. An aliquot (about 1/10th of the pool) was removed and neutralized with 1 M Tris, pH 7.0. The volume of Tris used was about ⅕^{th} the volume of the aliquot. The two pools were quickly frozen in a dry ice/alcohol bath and lyophilized.

The neutralized aliquots from each pool were buffer exchanged with PBS pH 7.4, 0.02% azide, then analyzed with SEC. It appears that the majority of protein eluted from the L243 column is contained in Pool 2. The MW of the SEC peaks were >400 kDa, 256 kDa, 119 ka, 58 kDa and 12 kDa.

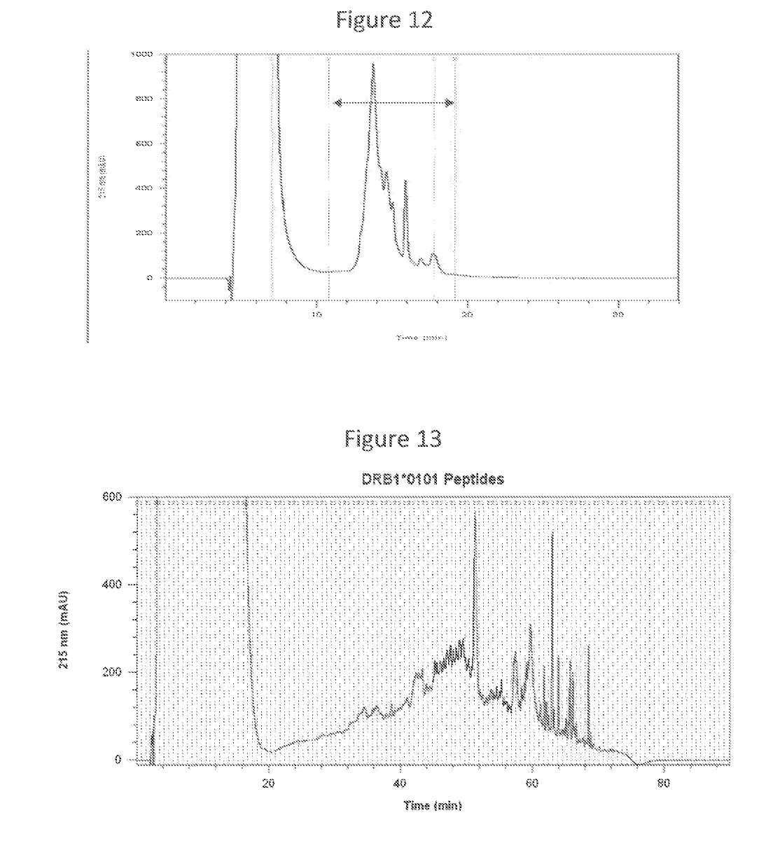

For isolation of the peptide cargo of the class II sHLA molecules, the lyophilized Pool 2 was dissolved in 10% acetic acid (80 mL) and heated to 76° C. for 20 min. After heating, the acetic acid solution was placed in the stirred cell with a 10 kDa membrane. The material that passed through the membrane was collected and lyophilized (125 mL). After lyophilization, the tubes were thoroughly rinsed with two 1 mL portions of 10% acetic acid. The acetic acid solution was concentrated to approximately 1 mL, and 100 mL was desalted using the RP-HPLC (FIG. 12). The solvent was removed from the pooled fractions, and the sample was submitted for Edman degradation. The remaining 900 μl was separated using RP-HPLC (FIG. 13).

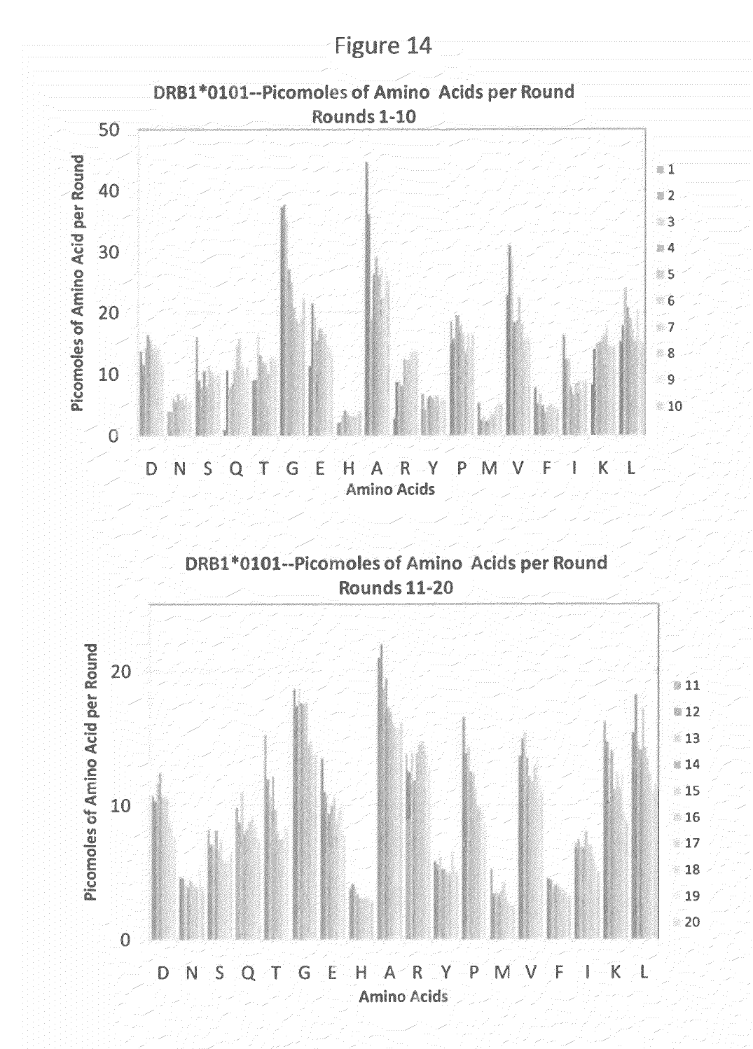

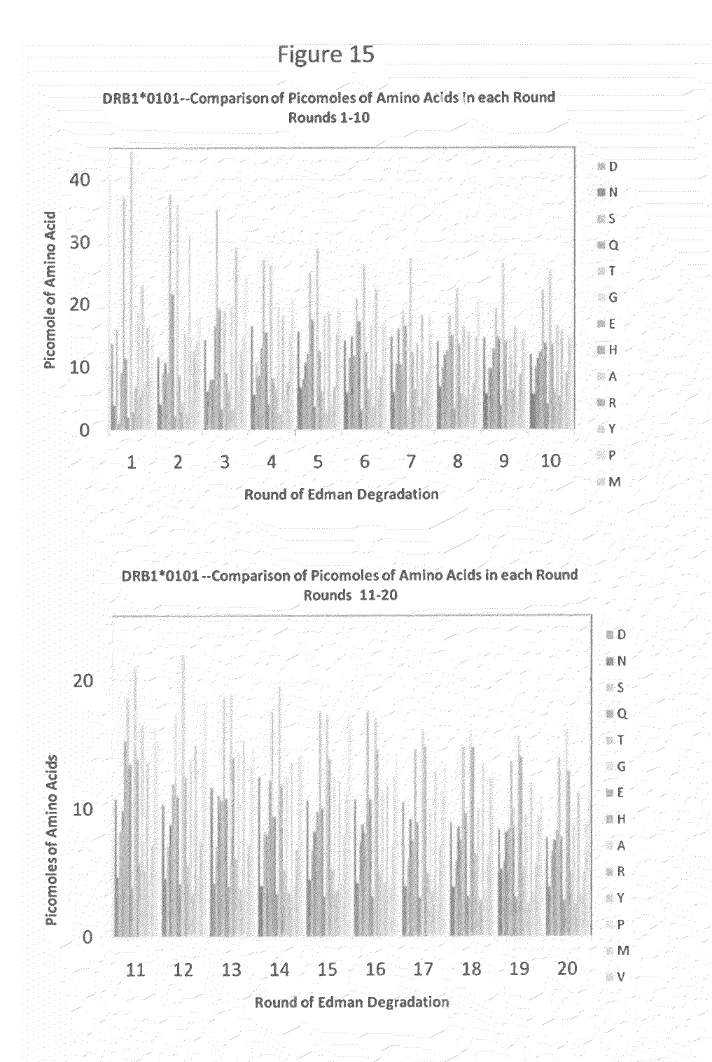

The Edman data presented in FIGS. 14 and 15 clearly demonstrate the presence of endogenously produced and loaded peptide ligands in the class II sHLA trimolecular complexes. FIG. 14 graphically depicts how each amino acid changes with each round of Edman degradation. FIG. 15 compares picomoles of amino acids in each cycle.

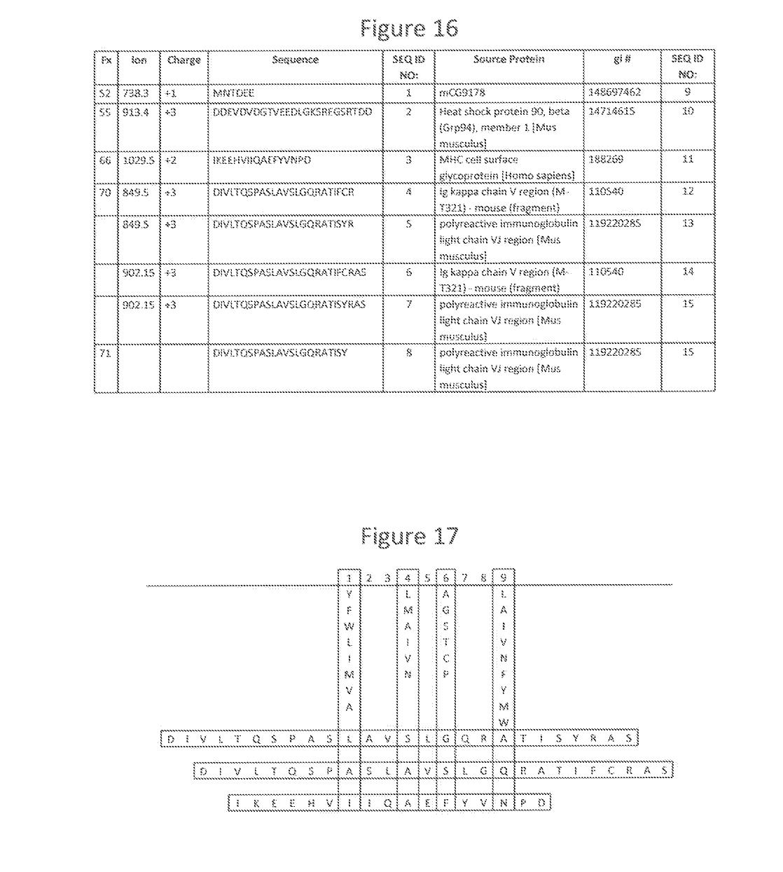

In addition, FIG. 16 contains a table of peptides identified by the methods described herein above. FIG. 17 contains potential alignments of three of these sequences with the common peptide motif for DRB1*0101. These data confirm that peptides isolated from sHLA DRB1*0101 are consistent with peptides characterized from cell surface associated DRB1*0101 molecules. Thus, the sHLA class II molecules represent a facile and biologically accurate tool to discover peptides restricted and presented by particular HLA class II alleles.

Thus, this Example demonstrates that the sHLA class II of the presently disclosed and claimed inventive concept(s) binds authentic restricted peptides that share the same core motif as that defined by native cell surface associated HLA class II.

### Example 3

### Use of Class II sHLA for HLA Specific Antibody Detection

Monitoring of soluble HLA class II production from cells was completed by a sandwich ELISA that uses the L243 antibody described in Example 2 to capture a class II complex (Lampson et al., 1980). In addition, an anti-leucine zipper monoclonal antibody was used as a product specific capture antibody to specifically monitor class II production. This antibody only recognizes conformationally intact leucine dimers that have been incorporated onto the ends of the HLA class II complex. For the ELISA, MaxiSorp™ StarWell™ plates (Nunc) were coated with 10 μg/ml mAb anti-leucine zipper in Carbonate Buffer. Samples were applied in tissue culture supernatant, captured with bound anti-leucine zipper mAb, and detected with a biotinylated mouse anti-human HLA-DR (One Lambda), conjugated to Avidin/Biotinylated Enzyme Complex (Vectorlabs). Samples were colorimetrically detected using the peroxidase substrate OPD (Sigma) and read on a standard plate reader at a wavelength of 490 nm. All samples were run in triplicate along with a sHLA class II complex standard that has been previously quantified by multiple methods. The ability of the sHLA class II to bind L243 demonstrates one enablement of the presently disclosed and claimed inventive concept(s) as a method to detect anti-HLA class II antibodies.

The immobilization of class II using antibodies can be utilized to build a sera screening kit for transplantation by placing each of the many different sHLA class II complexes produced into individual wells and then applying patient sera to every well as described above. The positive wells indicate the presence of patient antibodies to a particular class II molecule, and such antibodies can mediate acute graft rejection. Detection of antibodies to class II is important for transplant success. One skilled in the art of protein immobilization can envision methods other than the use of an antibody to immobilize class II for screening patient antibodies. More complex assays have been developed where a multiplex format with all antigens are in a single mixture. The use of a specific combination of dyes allows the antigens binding HLA specific antibodies to differentiate as positive.

### Example 4

### Use of Class II sHLA for Epitope Discovery

The presently disclosed and claimed inventive concept(s) is also related to methods of epitope discovery and comparative ligand mapping which can be utilized to distinguish diseased cells (i.e., infected or tumor cells) from non-diseased cells (i.e., uninfected or non-tumor cells) by unique epitopes presented by HLA molecules in the disease or non-disease state. The present Example is directed to said methods.

Approximately 25 mg of class II sHLA produced and purified as described herein above in Examples 1 and 2 (said quantity being measured by anti-leucine zipper dimer ELISA) was gathered from naïve and infected cells and passed over a Pharmacia XK-50 (Amersham-Pharmacia Biotech) column packed with 50 ml SEPHAROSE® Fast Flow 4B matrix (Amersham) coupled to L243 antibody. Bound class II complexes were washed first with 1 L 20 mM sodium phosphate wash buffer. After washing, peptides were eluted with freshly made 0.2 N acetic acid, pH 2.7.

Peptide-containing eluate fractions were brought up to 10% glacial acetic acid concentration and were heated in a 76-78° C. water bath for 10 minutes to denature HLA class II alpha and beta chains and free peptides. Fractions were pooled into an ultrafiltration device containing a 3 kDa molecular-weight cutoff membrane. Peptides were collected and lyophilized to dryness. Peptides were resuspended in 10% acetic acid; 10% of the peptide pool was then purified through a first-round of C12 HPLC with an acetonitrile gradient of 2-80% over 5 minutes and then hold at 80% for 4 minutes, with 10-minute fractions collected. The peptide-containing fractions were pooled, speed-vacuumed to dryness, and resuspended in 10% acetic acid. This fraction was used for 14 rounds of Edman degradation sequencing to demonstrate that peptides were eluted from the class II sHLA of interest. The remaining 90% of the peptides were fractionated by RP-HPLC using an acetonitrile gradient of 2-10% over 2 minutes followed by 10-60% over 60 minutes, with 0.7-minute fractions collected. Peptides eluted in a given fraction were monitored by UV absorbance at 216 nm.

Fractionated peptides were mapped by MS to generate fraction-based ion maps. Fractions were speed-vacuumed to dryness and resuspended in 20 μl 50:50 methanol:water plus 0.05% acetic acid. Then 1 μl was removed and sprayed via nanoelectrospray (Proxeon) into a Q-STAR® Elite quadrupole mass spectrometer with a time-of-flight (TOF) detector (ABI SCIEX). Spectra were generated for masses in the range of 300-1200 amu using identical mass spectrometer settings for each fraction sprayed. Spectra were then baseline subtracted and analyzed by using either BioMultiview version 1.5beta9 (ABI SCIEX) or BioAnalyst version 2.0 (ABI SCIEX). Spectra from the same fraction in uninfected/infected cells were manually aligned to the same mass range, locked, and 15 amu increments visually assessed for the presence of differences in the ions represented by the spectra as demonstrated in a sHLA class I model. Ions were selected for MS/MS sequencing that are unique to infected-cell MS ion spectra or are upregulated 1.5 fold over the same ion in the corresponding uninfected-cell MS ion spectra. Ions are thus categorized into multiple categories before MS/MS sequencing.

Ions masses unique to infected cells and upregulated in infected cells were subjected to MS/MS sequencing. HPLC fractions containing peptides to be sequenced were sprayed into the mass spectrometer in 1 μl aliquots. All MS/MS settings were kept constant except for the Q2 collision energy and Cad gas settings, which were varied to achieve the best fragmentation. Fragmentation patterns generated were interpreted manually and with the aid of BioMultiView version 1.5beta9. Multiple, free, web-based applications were used to automate peptide identification including MASCOT, Protein Prospector, and BLAST search.

Identified epitopes were validated before they were categorized as unique or upregulated. First, in the corresponding uninfected HPLC fraction and one fraction before and after, the amu of the putative peptide undergoes MS/MS under the same fragmentation conditions as demonstrated in a sHLA class I model. Next, the spectra from uninfected and infected cells were overlaid to ensure that the putative peptide is truly unique or increased. Second, synthetic peptides were generated for each influenza peptide identified. These synthetic peptides were resuspended in 10% acetic acid and RP-HPLC fractionated under the same conditions as employed for the original fractionation, ensuring that the peptide putatively identified has the same hydrophobicity as that of the ion MS/MS fragmented. This synthetic peptide was MS/MS fragmented under the same collision conditions as that of the ion. Then, the spectra were overlaid, and checked for an exact match with the original peptide fragment.

### Example 5

### Use of Class II sHLA for Antibody Removal

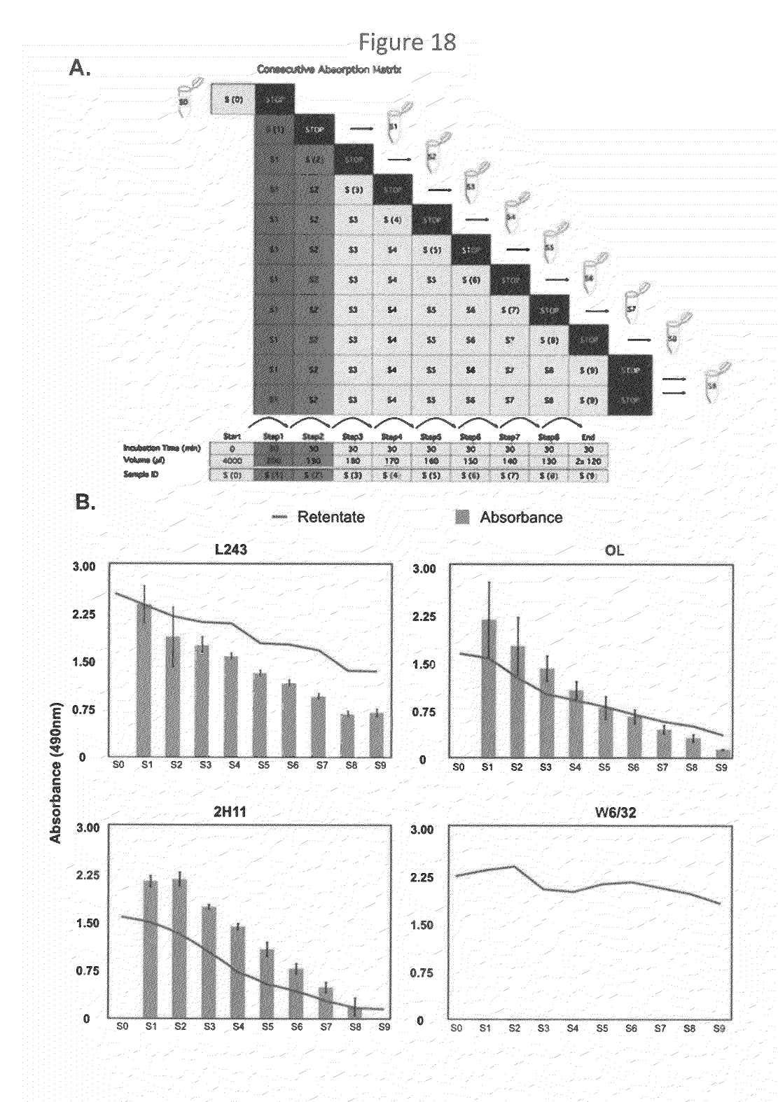

The soluble HLA class II trimolecular complexes of the presently disclosed and claimed inventive concept(s) have also been demonstrated herein to be successfully used in antibody removal techniques, as illustrated in FIGS. 18-22.

FIG. 18 graphically depicts coupling of soluble DRB1*1101 ZP HLA Class II trimolecular complex to a solid support and use thereof to facilitate removal of HLA Class II specific antibodies in an ELISA format. Panel A contains a diagram of the consecutive absorption matrix ELISA performed for specific antibody removal. Briefly, soluble HLA Class II DRB1*1101/DRA1*0101 ZP (labeled as DRB1*1101) was coated to a standard ELISA plate and blocked with BSA. Biotinylated labeled HLAII specific antibodies were then prepared and diluted according to a pre-determined titration for optimal binding, and added to 10 wells as S1. A small portion of this original dilution (204.1) was saved as S(0). The antibody was allowed to bind for 30 minutes at room temperature, after which the entire contents of each well (<200 μl) was moved to a corresponding new well (S2), and BSA buffer was added to the S1 wells. This entire process was repeated for a total of 9 sample rounds (S1-S9). For each round, one well was saved in an eppendorf tube for evaluation of the amount of antibody remaining in the retentate solution. These were marked as S(n). After the absorption process was completed, the plate was developed using HRP/OPD peroxidase substrate and plotted as “absorbance.” The retentate samples were also read on a separate ELISA plate in the same manner. These were plotted as “retentate.” Panel B depicts absorbance and retentate values from 3 different HLA Class II specific mAb antibodies: L243, OL (One Lambda), and 2H11 were subjected to the consecutive absorbance matrix. The L243 and OL mAbs, specific for the HLA Class II molecules, and the 2H11 mAb, specific for the zipper tail piece recombinantly added to the soluble HLA Class II molecules, showed a reduction of HLA class II antibodies in the absorption and retentate through each round of the ELISA. One control mAb antibody was included, W6/32, which is specific for HLA Class I molecules, which was not absorbed to the plate and only found in the retentate.

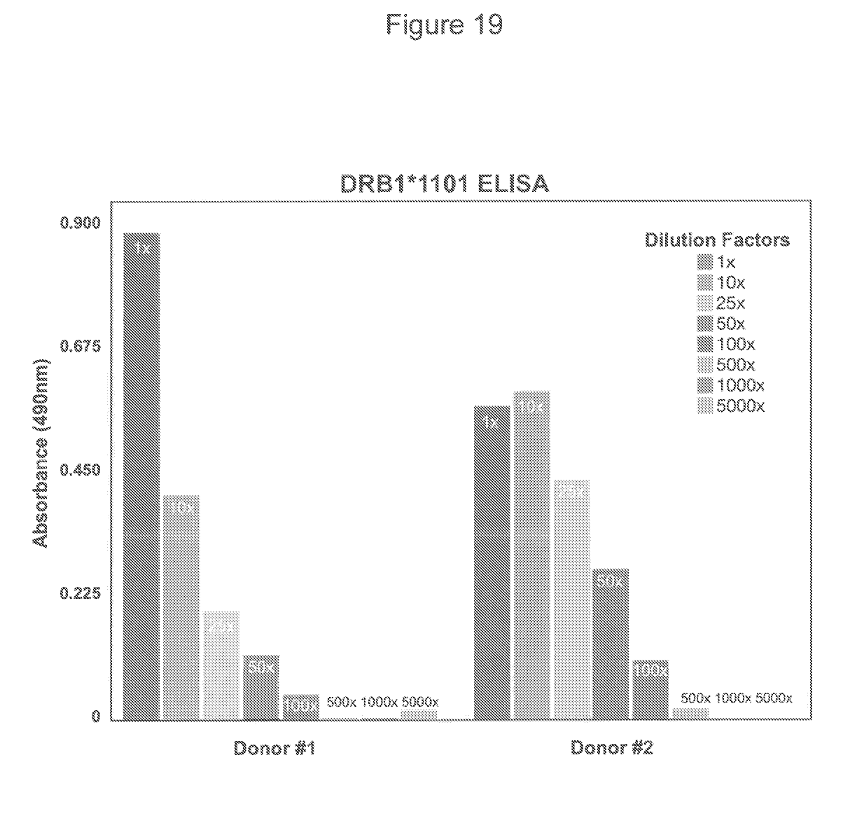

FIG. 19 graphically depicts that DRB1*1101-specific human sera was recognized by soluble DRB1*1101 in an ELISA format. Using soluble HLA Class II DRB1*1101/DRA1*0101 ZP (labeled as DRB1*1101), ELISA plates were directly coated with the HLA Class II soluble allele. Serum samples from two human donors known previously to have DRB1*1101 reactivity were added to the plates in a dilution range from 1× (no dilution) to 5000×. Plates were washed, and a secondary biotinylated goat anti-human IgG antibody was added. Plates were developed using HRP/OPD peroxidase substrate and read at absorbance of 490 nm. Dilution curves for the sera antibody reactivity can be seen for both donors, corresponding to specific avidity for DRB1*1101.

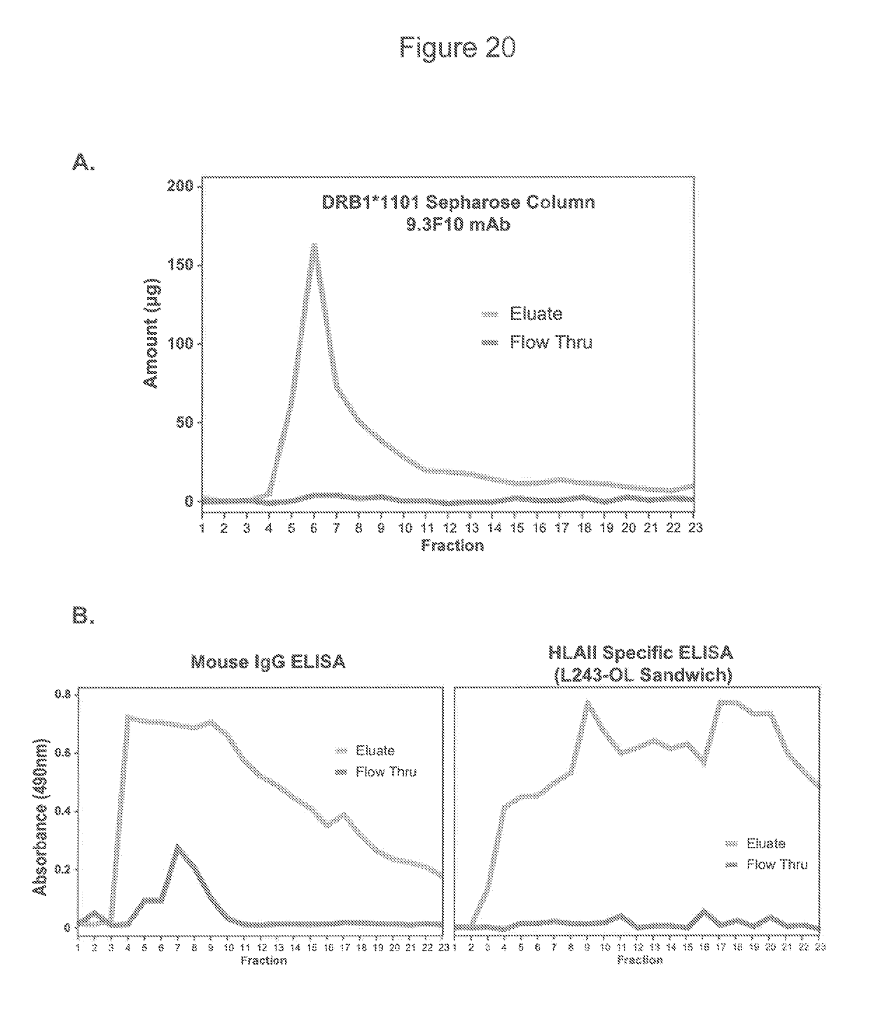

FIG. 20 graphically depicts that soluble DRB1*1101 can be coupled to SEPHAROSE® and used to absorb the HLA Class II specific antibody, 9.3F10. In Panel A, 4 mg of soluble DRB1*1101 was coupled to 1 ml of FastFlow SEPHAROSE® and packed into a gravity column. A known mixture of 100 μg/ml of mAb 9.3F10 (in 1×PBS), which has DR reactivity, was passed over the column and washed with 1×PBS. A total of 23 200 μl fractions of flow thru were collected, weighed, and measured for OD 280 nm. Values were converted to total amount of protein. To elute the column, roughly 4 ml of DEA (diethanolamine) buffer, pH 11.3, was added to the column, and fractions were collected in 200 μl quantities. The eluate was also weighed, measured at an optical density of 280 nm, and converted to total amount of protein.

In Panel B of FIG. 20, two separate ELISAs for total mouse IgG and human HLA were also performed on the Flow Thru and Eluate to detect specific antibodies (versus HLA proteins) that might have been eluted off the column. Due to the increase in ELISA sensitivity, the minuscule amount of protein seen in the flow thru gave a small peak in the antibody ELISA. Importantly, however, no HLA was seen in the flow thru, but HLA did elute off the column when DEA was added.

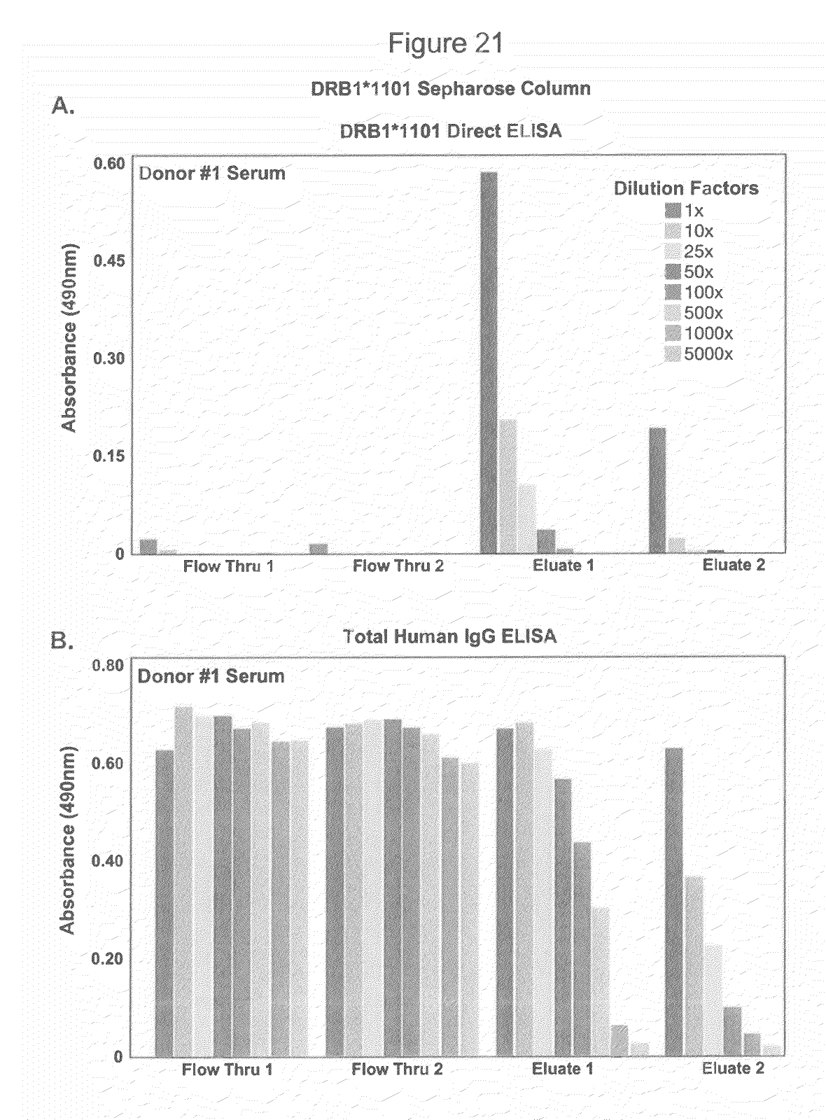

FIG. 21 graphically depicts that antibodies contained in human sera specific for DRB1*1101 can be removed by a DRB1*1101 specific column. Donor #1 sera was passed over the DRB1*1101 SEPHAROSE® column, and two 2 ml fractions of flow thru were collected. To elute, DEA buffer, pH 11.3 was added to the column, and two 2 ml fractions were collected. In Panel A, a direct DRB1*1101 ELISA was performed to detect the amount of DRB1*1101 specific antibodies that were left in the flow thru and eluate. Flow thru and eluate fractions were diluted 1× (no dilution) to 5000× and developed with a biotinylated goat anti-human secondary antibody, followed by HRP/OPD peroxidase substrate. Plates were read at an optical density of 490 nm. In Panel B, a total human IgG sandwich ELISA was also performed to evaluate passage of total human IgG. Total human IgG was seen to pass thru; however only DRB1*1101 antibodies were retained by the column, and only seen once the column was eluted.

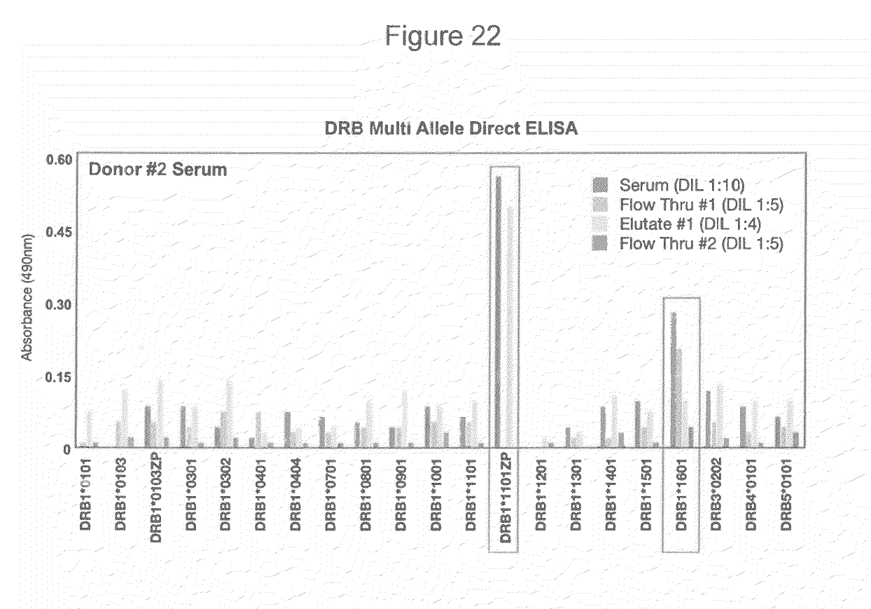

FIG. 22 graphically depicts that soluble DRB1*1101 coupled SEPHAROSE® is specific for DRB1*1101 and not other DR alleles. Donor #2 sera was passed over the same DR1*1101 column in the same manner as FIG. 21, and two fractions of the flow thru and one fraction of the eluate were evaluated for multi-allele DR reactivity. Briefly, multiple alleles of DR from membrane detergent purifications and two DR alleles produced solubly were coated to a 96 well ELISA plate in previously determined optimal amounts for reactivity. Two flow thru fractions and one of the eluate fractions were compared to the original sera sample for reactivity. The second eluate fraction was not evaluated given that most of the specific reactivity was contained in Eluate #1 (FIG. 21). Low reactivity was seen across the board except for the soluble DRB1*1101 (DRB1*1101 ZP) allele, which gave high reactivity to only the sera sample and the eluate but not the flow thrus (first boxed area). The sera also contained strongly reactive antibodies to a second allele, DRB1*1601 (second boxed area), which passed through the flow thru but not the eluate.

Therefore, this Example demonstrates that sHLA class II trimolecular complexes immobilized in a column format can selectively and efficiently remove the vast majority of anti-HLA specific antibodies based on affinity to the bound HLA class II protein in a single pass through, while not removing antibodies that bind to antigenically dissimilar HLA molecules. These data show that a highly specific and efficient antibody removal device can be constructed using the sHLA class II proteins produced in accordance with the presently disclosed and claimed inventive concept(s).

Thus, in accordance with the presently disclosed and claimed inventive concept(s), there have been provided methods of producing soluble HLA class II trimolecular complexes, and methods of use thereof, that fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.
Adams, S. 2000. Nat Med 6:337-342.
Bohmig, G. A., et al. 2000. Am J Kidney Dis 35:667-673.
Chang, H., et al. 1994. Immunology, 91:11408-11412.
Crawford, F., et al. 1998. Immunity, 8:675-682.
Ditschkowski, M., et al. 1999. Ann Surg. 229(2): 246-254.
Emonds, M. P., et al. 2000. Pediatr Transplant, 4:6-11.
Gaseitsiwe and Maeurer, 2009. Methods in Mol. Bio. 524:417-26.
Gronski and Weinem, 2006. Rev. Diabet. Stud. 3:88-95.
Guerra, C. B., et al. 1998. J. of Immunol., 160: 4289-4297.
Hansen, John A. 2005. Biology of Blood and Marrow Transplantation 11:24-27.
Herold et al., 2009. Clin Immunol. 132:166-173.
Hickman, H., et al. 2003. J. of Immunology, 171: 22-26.
Howden, A. J., et al. 2000. Hum Immunol, 61:419-24.
Jones et al., 2006. Nat. Rev. Immunol. 6:271-282.
Kalandadze, A., et al. 1996. J. Bio. Chem. 271:20156-20162.
Kaufman and Herold. 2009. Diabetes Metab. Res. Rev. 25:302-6.
Kezuka, T., et al., 2001. Arch Ophthalmol. 119(7):1044-9.
Lampson, L. and Levy, R. 1980. J. Immunol. 125: 293-299.
Landschulz, W., et al. 1988. Science, Vol. 240.
McMurtrey, C., et al. 2008. PNAS, 105:2981-2986.
Muller-Steinhardt, M., et al. 2000. Clin Transplant, 14:85-9.
Nepom and Kwok, 1998. Diabetes, 47:1177-84.
Novak, E., et al. 1999. J. Clin. Invest. 104:R63-R67.
Pratesi, F., et al. 2000. J Rheumatol, 27:109-15.
Prilliman, K. R., et al. 1999. J. Immunology, 162:7277.
Schuna, A. A. and C. Megeff. 2000. Am J Health Syst Pharm, 57:225-34. 2000.
Streilein, J W et al. 2007. Ocul Immunol Inflamm. 15(3):187-94.
Todd et al., 1988. Science, 240:1003-1009.
Weber et al., 2007. Oncologist. 12:864-72.
Wettstein, D. A., et al. 1991. J. Exp. Med. 174:219-228.
Wicker et al., 1996. J. Clin. Invest. 98:2597-2603.
Wilson, B., 1981. Scand. J. Immunol., 14:201-205.
Yoon and Jun, 2001. Ann N Y Acad. Sci. 928:200-11.
Yoshida and Kikutani, 2000. Rev. Immunogenet. 2:140-6.
Zimmer, K. P., et al. 1995. Gut. 36: 703-709.

What is claimed is:

1. A method of producing isolated, HLA class II trimolecular complexes, wherein the isolated, HLA class II trimolecular complexes comprise a soluble, glycosylated alpha chain, a soluble, glycosylated beta chain, and a non-covalently associated, endogenously produced peptide ligand, the method comprising the steps of:
inserting a first isolated nucleic acid segment and a second isolated nucleic acid segment into a mammalian cell line, the first isolated nucleic acid segment encoding a soluble form of an alpha chain of a HLA class II molecule having a first domain of a super secondary structural motif attached thereto, and the second isolated nucleic acid segment encoding a soluble form of a beta chain of the HLA class II molecule having a second domain of the super secondary structural motif attached thereto, wherein the mammalian cell line is a non-human mammalian cell line or a human cell line that does not express endogenous HLA class II, and wherein the mammalian cell line comprises glycosylation mechanisms required for glycosylation of proteins produced therein and chaperone complexes required for peptide ligand loading into HLA class II molecules;
culturing the recombinant mammalian cell line under conditions that allow for expression of the soluble class II alpha and beta chains, association of the soluble class II alpha and beta chains through the first and second domains of the super secondary structural motif, glycosylation of the soluble class II alpha and beta chains, and loading of an antigen binding groove formed from the soluble class II alpha and beta chains with an endogenously produced, non-covalently associated peptide ligand, thereby producing soluble class II trimolecular complexes; and
isolating the soluble class II trimolecular complexes secreted from the recombinant mammalian cell line, whereby each trimolecular complex so isolated comprises identical recombinant, individual soluble alpha and beta chain molecules of the HLA class II.

2. The method of claim 1, wherein the first and second isolated nucleic acid segments are present in a single recombinant vector.

3. The method of claim 1, wherein the first isolated nucleic acid segment is present in a first recombinant vector and the second isolated nucleic acid segment is present in a second recombinant vector.

4. The method of claim 1, wherein the super secondary structural motif attached to the alpha and beta chains is a leucine zipper motif sequence that acts as a tethering moiety for the alpha and beta chains.

5. A method of producing functionally active, individual soluble HLA class II trimolecular complexes that are purified substantially away from other proteins such that the individual soluble HLA class II trimolecular complexes maintain the physical, functional and antigenic integrity of the native HLA class II trimolecular complex, wherein each trimolecular complex comprises a recombinant, soluble alpha chain and a recombinant, soluble beta chain of an individual HLA class II molecule, and a peptide endogenously loaded in an antigen binding groove formed by the alpha and beta chains of the individual soluble HLA class II molecule, the method comprising the steps of:
inserting a first isolated nucleic acid segment and a second isolated nucleic acid segment into a mammalian cell line, the first isolated nucleic acid segment encoding a soluble form of an alpha chain of a HLA class II molecule having a first domain of a super secondary structural motif attached thereto, and the second isolated nucleic acid segment encoding a soluble form of a beta chain of the HLA class II molecule having a second domain of the super secondary structural motif attached thereto, wherein the mammalian cell line is a non-human cell line or a human cell line that does not express endogenous HLA class II, and wherein the mammalian cell line comprises glycosylation mechanisms required for glycosylation of proteins produced therein and chaperone complexes required for peptide ligand loading into HLA class II molecules;
culturing the recombinant mammalian cell line under conditions that allow for expression of the soluble class II alpha and beta chains, association of the soluble class II alpha and beta chains through the first and second domains of the super secondary structural motif, glycosylation of the soluble class II alpha and beta chains, and loading of an antigen binding groove formed from the soluble class II alpha and beta chains with an endogenously produced, non-covalently associated peptide ligand, thereby producing soluble HLA class II trimolecular complexes; and
purifying the individual, soluble HLA class II trimolecular complexes substantially away from other proteins, wherein the individual soluble HLA class II trimolecular complexes maintain the physical, functional and antigenic integrity of the native HLA class II trimolecular complex, and wherein each trimolecular complex so purified comprises identical recombinant, individual soluble alpha and beta chain molecules of the HLA class II.

6. The method of claim 5, wherein the first and second isolated nucleic acid segments are present in a single recombinant vector.

7. The method of claim 5, wherein the first isolated nucleic acid segment is present in a first recombinant vector and the second isolated nucleic acid segment is present in a second recombinant vector.

8. The method of claim 5, wherein the super secondary structural motif attached to the alpha and beta chains is a leucine zipper motif sequence that acts as a tethering moiety for the alpha and beta chains.

9. A multimer of at least two soluble HLA class II trimolecular complexes, wherein each of the at least two soluble HLA class II trimolecular complexes comprises a soluble, glycosylated alpha chain attached to a soluble, glycosylated beta chain via a super secondary structure, and a non-covalently associated, endogenously produced peptide ligand disposed in an antigen binding groove formed by the association of the alpha and beta chains.

10. The multimer of claim 9, wherein the at least two soluble HLA class II trimolecular complexes comprises a tail attached thereto to aid in multimerization.

11. The multimer of claim 9, wherein the tail is selected from the group consisting of a biotinylation signal peptide tail, an immunoglobulin heavy chain tail, a TNF tail, an IgM tail, a leucine zipper, a Fos/Jun tail, and combinations thereof.


SEQUENCE LISTING (21 sequences)

SEQ ID NO: 1 - protein, 6 aa, Mus musculus
MNTDEE

SEQ ID NO: 2 - protein, 25 aa, Mus musculus
DDEVDVDGTVEEDLGKSREGSRTDD

SEQ ID NO: 3 - protein, 17 aa, Homo sapiens
IKEEHVIIQAEFYVNPD

SEQ ID NO: 4 - protein, 24 aa, Mus musculus
DIVLTQSPASLAVSLGQRATIFCR

SEQ ID NO: 5 - protein, 24 aa, Mus musculus
DIVLTQSPASLAVSLGQRATISYR

SEQ ID NO: 6 - protein, 26 aa, Mus musculus
DIVLTQSPASLAVSLGQRATIFCRAS

SEQ ID NO: 7 - protein, 26 aa, Mus musculus
DIVLTQSPASLAVSLGQRATISYRAS

SEQ ID NO: 8 - protein, 23 aa, Mus musculus
DIVLTQSPASLAVSLGQRATISY

SEQ ID NO: 9 - protein, 292 aa, Mus musculus
MFYAVLGIPLTLVMFQSLGERMNTFVRYLLKRIKKCCGMRNTEVSMENMVTVGFFSCMGTLCLGAAAFSQCEDWSFFHAYYYCFITLTTIGFGDFVALQAKGALQRKPFYVAFSFMYILVGLTVIGAFLNLVVLRFLTMNTDEELLEGEVAEILAGNPRRVSVRAPQRRKRHHAMYFLRKYGRTLCYLCFPGTNWGKDDDDDDDDDVVDNVVVTAPISAPAPAPAPAPAPAAVAAGATIRSVRATVHTVSCRVEEIPPDVLRNTYFRSVFGAIPPGMHTCGDHRLHLRRKSI

SEQ ID NO: 10 - protein, 802 aa, Mus musculus
MRVLWVLGLCCVLLTFGFVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVRKYSQFINFPIYVWSSKTETVEEPLEEDEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHSTDITSLDQYVERMKEKQDKIYFMAGSSRKEAESSPFVERLLKKGYEVIYLTEPVDEYCIQALPEFDGKRFQNVAKEGVKFDESEKTKESREATEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVMDLAVVLFETATLRSGYLLPDTKAYGDRIERMLRLSLNIDPEAQVEEEPEEEPEDTSEEAEDSEQDEGEEMDAGTEEEEEETEKESTEKDEL

SEQ ID NO: 11 - protein, 229 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 12 - protein, 112 aa, Mus musculus
DIVLTQSPASLAVSLGQRATIFCRASQSVDYNAISYMHWYQQKPGQPPKLLIYAAANLESGIPARFSGSGSGTDFTLDIHPVEEEDAATYYCQQSSEDPYTFGGGTKLEIKR

SEQ ID NO: 13 - protein, 108 aa, Mus musculus
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 14 - protein, 112 aa, Mus musculus; identical to SEQ ID NO: 12

SEQ ID NO: 15 - protein, 108 aa, Mus musculus; identical to SEQ ID NO: 13

SEQ ID NO: 16 - DNA, 786 bp, artificial sequence
  construct encoding truncated, soluble human DRA1*0101 fused to linker and leucine zipper motif
atggccataagtggagtccctgtgctaggatttttcatcatagctgtgctgatgagcgctcaggaatcatgggctdraatcaaagaagaacatgtgatcatccaggccgagttctatctgaatcctgaccaatcaggcgagtttatgtttgacdratttgatggtgatgagattttccatgtggatatggcaaagaaggagacggtctggcggcttgaagaatttggacgadratttgccagctttgaggctcaaggtgcattggccaacatagctgtggacaaagccaacctggaaatcatgacaaagdracgctccaactatactccgatcaccaatgtacctccagaggtaactgtgctcacgaacagccctgtggaactgagadragagcccaacgtcctcatctgtttcatcgacaagttcaccccaccagtggtcaatgtcacgtggcttcgaaatggadraaaacctgtcaccacaggagtgtcagagacagtcttcctgcccagggaagaccaccttttccgcaagttccactatdractccccttcctgccctcaactgaggacgtttacgactgcagggtggagcactggggcttggatgagcctcttctcdraaagcactgggagtttgatgctccaagccctctcccagagactacagaggtcgacggaggaggaggtggagctcagctcgaaaaagagctccaggccctggagaaggaaaatgcacagctggaatgggagttgcaagcactggaaaaggaactggctcagtga

SEQ ID NO: 17 - protein, 253 aa, artificial sequence
  amino acid sequence encoded by SEQ ID NO16
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGAQLEKELQALEKENAQLEWELQALEKELAQ

SEQ ID NO: 18 - DNA, 822 bp, artificial sequence
  construct encoding truncated, soluble human DRB1*040101 fused to a linker and leucine zipper motif
atggtgtgtctgaagttccctggaggctcctgcatggcagctctgacagtgacactgatggtgctgagctccccadrbctggctttggctggggacacccgaccacgtttcttggagcaggttaaacatgagtgtcatttcttcaacgggacgdrbgagcgggtgcggttcctggacagatacttctatcaccaagaggagtacgtgcgcttcgacagcgacgtgggggagdrbtaccgggcggtgacggagctggggcggcctgatgccgagtactggaacagccagaaggacctcctggagcagaagdrbcgggccgcggtggacacctactgcagacacaactacggggttggtgagagcttcacagtgcagcggcgagtctatdrbcctgaggtgactgtgtatcctgcaaagacccagcccctgcagcaccacaacctcctggtctgctctgtgaatggtdrbttctatccaggcagcattgaagtcaggtggttccggaacggccaggaagagaagactggggtggtgtccacaggcdrbctgatccagaatggagactggaccttccagaccctggtgatgctggaaacagttcctcggagtggagaggtttacdrbacctgccaagtggagcacccaagcctgacgagccctctcacagtggaatggagagcacggtctgaatctgcacagdrbagcaaggtcgacggaggaggaggtggagctcagttgaaaaagaaattgcaagcactgaagaaaaagaacgctcagctgaagtggaaacttcaagccctcaagaagaaactcgcccagtga

SEQ ID NO: 19 - protein, 264 aa, artificial sequence
  amino acid sequence encoded by SEQ ID NO18
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKVDGGGGGAQLKKKLQALKKKNAQLKWKLQALKKKLAQ

SEQ ID NO: 20 - DNA, 825 bp, artificial sequence
  construct encoding truncated, soluble human DRB1*0103 fused to a linker and leucine zipper motif
atggtgtgtctgaagctccctggaggctcctgcatgacagcgctgacagtgacactgatggtgctgagctccccadrbctggctttggctggggacacccgaccacgtttcttgtggcagcttaagtttgaatgtcatttcttcaatgggacgdrbgagcgggtgcggttgctggaaagatgcatctataaccaagaggagtccgtgcgcttcgacagcgacgtgggggagdrbtaccgggcggtgacggagctggggcggcctgatgccgagtactggaacagccagaaggacatcctggaagacgagdrbcgggccgcggtggacacctactgcagacacaactacggggttggtgagagcttcacagtgcagcggcgagttgagdrbcctaaggtgactgtgtatccttcaaagacccagcccctgcagcaccacaacctcctggtctgctctgtgagtggtdrbttctatccaggcagcattgaagtcaggtggttccggaacggccaggaagagaaggctggggtggtgtccacaggcdrbctgatccagaatggagattggaccttccagaccctggtgatgctggaaacagttcctcggagtggagaggtttacdrbacctgccaagtggagcacccaagtgtgacgagccctctcacagtggaatggagagcacggtctgaatctgcacagdrbagcaaggtcgacggaggaggaggtggagctcagttgaaaaagaaattgcaagcactgaagaaaaagaacgctcagdrbctgaagtggaaacttcaagccctcaagaagaaactcgcccagtga

SEQ ID NO: 21 - protein, 264 aa, artificial sequence
  amino acid sequence encoded by SEQ ID NO20
MVCLKLPGGSCMTALTVTLMVLSSPLALAGDTRPRFLWQLKFECHFFNGTERVRLLERCIYNQEESVRFDSDVGEYRAVTELGRPDAEYWNSQKDILEDERAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKVDGGGGGAQLKKKLQALKKKNAQLKWKLQALKKKLAQ